(19) United States
(12) Patent Application Publication
(10) Pub. No.: US 2024/0221366 A1
(43) Pub. Date: Jul. 4, 2024

(54) LEARNING MODEL GENERATION METHOD, IMAGE PROCESSING APPARATUS, INFORMATION PROCESSING APPARATUS, TRAINING DATA GENERATION METHOD, AND IMAGE PROCESSING METHOD

(71) Applicant: TERUMO KABUSHIKI KAISHA, Tokyo (JP)

(72) Inventors: Shunsuke YOSHIZAWA, Ebina-shi (JP); Yasukazu SAKAMOTO, Hiratsuka-shi (JP); Katsuhiko SHIMIZU, Fujinomiya-shi (JP); Hiroyuki ISHIHARA, Tokyo (JP)

(73) Assignee: TERUMO KABUSHIKI KAISHA, Tokyo (JP)

(21) Appl. No.: 18/606,892

(22) Filed: Mar. 15, 2024

Related U.S. Application Data
(63) Continuation of application No. PCT/JP2022/034448, filed on Sep. 14, 2022.

(30) Foreign Application Priority Data
Sep. 17, 2021 (JP) 2021-152459

(51) Int. Cl. G06V 10/774; A61B 1/00; G06V 10/26; G06V 10/44; G06V 10/764

(52) U.S. Cl. CPC G06V 10/774; A61B 1/000096; G06V 10/267; G06V 10/457; G06V 10/764; G06V 2201/03

(57) ABSTRACT

A learning model generation method for generating a learning model that aids understanding of an image acquired with an image-acquiring catheter. The learning model generation method includes: creating a division line that divides a lumen region into a first region into which the image-acquiring catheter is inserted and a second region reaching an edge of a two-dimensional image, when it is determined that the lumen region reaches an edge of the two-dimensional image; creating second classification data in which a probability of being the lumen region and a probability of being an extra-luminal region are allocated; recording the two-dimensional image associated with the second classification data in a training database; and generating a learning model that outputs third classification data in which an input two-dimensional image is classified into a plurality of regions including a living tissue region, the lumen region, and an extra-luminal region, by machine learning.

### CROSS-REFERENCES TO RELATED APPLICATIONS

This application is a continuation of International Application No. PCT/JP2022/034448 filed on Sep. 14, 2022, which claims priority to Japanese Application No. 2021-152459 filed on Sep. 17, 2021, the entire content of both of which is incorporated herein by reference.

### TECHNOLOGICAL FIELD

The present disclosure generally relates to a learning model generation method, an image processing apparatus, an information processing apparatus, a training data generation method, and an image processing method.

### BACKGROUND DISCUSSION

A catheter system that acquires an image by inserting an image-acquiring catheter into a luminal organ such as a blood vessel has been used (International Patent Application Publication No. WO 2017/164071 A).

However, in an image acquired using an image-acquiring catheter, there are cases where part of information about the luminal organ is drawn in a missing state. In such an image with a defect, the structure of the luminal organ cannot be correctly visualized. Therefore, there are cases where it is difficult for the user to quickly understand the structure of the luminal organ.

### SUMMARY

A learning model generation method is disclosed, which is configured to aid the understanding of an image acquired with an image-acquiring catheter.

A learning model generation method includes: acquiring a two-dimensional image acquired with an image-acquiring catheter; acquiring first classification data in which the respective pixels constituting the two-dimensional image are classified into a plurality of regions including a living tissue region, a lumen region into which the image-acquiring catheter is inserted, and an extra-luminal region outside the living tissue region; determining, in the two-dimensional image, whether the lumen region reaches an edge of the two-dimensional image; when it is determined that the lumen region does not reach an edge of the two-dimensional image, associating the two-dimensional image with the first classification data, and recording the two-dimensional image associated with the first classification data in a training database; when it is determined that the lumen region reaches an edge of the two-dimensional image, creating a division line that divides the lumen region into a first region into which the image-acquiring catheter is inserted and a second region reaching an edge of the two-dimensional image; creating second classification data in which a probability of being the lumen region and a probability of being the extra-luminal region are allocated for each of small regions constituting the lumen region in the first classification data, on the basis of the division line and the first classification data; associating the two-dimensional image with the second classification data, and recording the two-dimensional image associated with the second classification data in the training database; and generating a learning model that outputs third classification data by machine learning using training data recorded in the training database when a two-dimensional image is input, the respective pixels constituting the two-dimensional image being classified into a plurality of regions including the living tissue region, the lumen region, and the extra-lumen region in the third classification data.

An image processing apparatus includes: an image acquisition unit configured to acquire a plurality of two-dimensional images obtained in time series with an image-acquiring catheter; a first classification data acquisition unit configured to acquire a series of first classification data in which respective pixels constituting each two-dimensional image of the plurality of two-dimensional images are classified into a plurality of regions including a living tissue region, a lumen region into which the image-acquiring catheter is inserted, and an extra-luminal region outside the living tissue region; a determination unit configured to determine whether the lumen region reaches an edge of each two-dimensional image, in each two-dimensional image of the plurality of two-dimensional images; a division line creation unit configured to create a division line that divides the lumen region into a first region into which the image-acquiring catheter is inserted and a second region reaching an edge of the two-dimensional image, when the determination unit determines that the lumen region reaches an edge of the two-dimensional image; and a three-dimensional image creation unit configured to create a three-dimensional image by using the series of first classification data in which a classification of the second region has been changed to the extra-luminal region, or by using the series of first classification data and processing the second region as the same region as the extra-luminal region.

An information processing apparatus includes: an image acquisition unit that acquires a two-dimensional image acquired with an image-acquiring catheter; a first classification data acquisition unit that acquires first classification data in which the two-dimensional image is classified into a plurality of regions including a living tissue region, a lumen region into which the image-acquiring catheter is inserted, and an extra-luminal region outside the living tissue region; a determination unit that determines, in the two-dimensional image, whether the lumen region reaches an edge of the two-dimensional image; a first recording unit that associates the two-dimensional image with the first classification data and records the two-dimensional image associated with the first classification data in a training database, when the determination unit determines that the lumen region does not reach an edge of the two-dimensional image; a division line creation unit that creates a division line that divides the lumen region into a first region into which the image-acquiring catheter is inserted and a second region reaching an edge of the two-dimensional image, when the determination unit determines that the lumen region reaches an edge of the two-dimensional image; a second classification data creation unit that creates second classification data in which a probability of being the lumen region and a probability of being the extra-luminal region are allocated for each of small regions constituting the lumen region of the first classification data, on a basis of the division line and the first classification data, when the determination unit determines that the lumen region reaches an edge of the two-dimensional image; and a second recording unit that associates the two-dimensional image with the second classification data, and records the two-dimensional image associated with the second classification data in the training database, when the determination unit determines that the lumen region reaches an edge of the two-dimensional image.

A training data generation method, includes: acquiring a two-dimensional image acquired with an image-acquiring catheter; acquiring first classification data in which the two-dimensional image is classified into a plurality of regions including a living tissue region, a lumen region into which the image-acquiring catheter is inserted, and an extra-luminal region outside the living tissue region; determining, in the two-dimensional image, whether the lumen region reaches an edge of the two-dimensional image; when it is determined that lumen region reaches an edge of the two-dimensional image, creating a division line that divides the lumen region into a first region into which the image-acquiring catheter is inserted and a second region reaching an edge of the two-dimensional image; creating second classification data in which a probability of being the lumen region and a probability of being the extra-luminal region are allocated for each of small regions constituting the lumen region of the first classification data, on a basis of the division line and the first classification data; and associating the two-dimensional image with the second classification data, and recording the two-dimensional image associated with the second classification data in a training database; and, when it is determined that the lumen region does not reach an edge of the two-dimensional image, associating the two-dimensional image with the first classification data, and recording the two-dimensional image associated with the first classification data in the training database.

An image processing method includes: acquiring a plurality of two-dimensional images obtained in time series with an image-acquiring catheter; acquiring a series of first classification data in which respective pixels constituting each two-dimensional image of the plurality of two-dimensional images are classified into a plurality of regions including a living tissue region, a lumen region into which the image-acquiring catheter is inserted, and an extra-luminal region outside the living tissue region; determining whether the lumen region reaches an edge of each two-dimensional image, in each two-dimensional image of the plurality of two-dimensional images; creating a division line that divides the lumen region into a first region into which the image-acquiring catheter is inserted and a second region reaching an edge of the two-dimensional image, when it is determined that the lumen region reaches an edge of the two-dimensional image; and creating a three-dimensional image by using the series of first classification data in which a classification of the second region has been changed to the extra-luminal region, or by using the series of first classification data and processing the second region as the same region as the extra-luminal region.

In one aspect, it is possible to provide a learning model generation method and the like configured to aid the understanding of an image acquired with an image-acquiring catheter.

### BRIEF DESCRIPTION OF THE DRAWINGS

FIG. 1 is a diagram for explaining a method for generating a third classification model.

FIG. 2 is a diagram for explaining first classification data.

FIG. 3 is a diagram for explaining the configuration of an information processing apparatus that creates a training database (DB).

FIG. 4 is a diagram for explaining the record layout in a first classification DB.

FIG. 5 is a diagram for explaining the record layout in the training DB.

FIG. 6 is a diagram for explaining a method for creating a division line.

FIG. 7 is a diagram for explaining a process to be performed in a case where an opening of a living tissue region is present at the end of an R-T format image in the theta direction.

FIG. 8 is a diagram for explaining second classification data.

FIG. 9A is a schematic diagram illustrating, in an enlarged manner, nine pixels at the place corresponding to a portion B in FIG. 8 in the first classification data.

FIG. 9B is an enlarged schematic diagram illustrating the nine pixels in the portion B in FIG. 8.

FIG. 10 is a diagram for explaining the second classification data.

FIG. 11 is a diagram for explaining the second classification data.

FIG. 12 is a diagram for explaining the second classification data.

FIG. 13 is a flowchart for explaining the flow of processing according to a program.

FIG. 14 is a flowchart for explaining the processing flow in a division line creation subroutine.

FIG. 15 is a flowchart for explaining the processing flow in a second classification data creation subroutine.

FIG. 16 is a diagram for explaining the configuration of an information processing apparatus that creates a third classification model.

FIG. 17 is a flowchart for explaining a flow of processing according to a program for machine learning.

FIG. 18 is a diagram for explaining an open/close determination model.

FIG. 19A is a diagram for explaining a state in which a plurality of candidate division lines is created for first classification data displayed in an R-T format.

FIG. 19B is a diagram for explaining a state in which FIG. 19A is coordinate-transformed into an X-Y format.

FIG. 20 is a flowchart for explaining the processing flow in a division line creation subroutine according to Modification 1-2.

FIG. 21 is a diagram for explaining candidate division lines according to Modification 1-4.

FIG. 22 is a diagram for explaining machine learning according to Modification 1-5.

FIG. 23 is a flowchart for explaining the flow of processing according to a program of a second embodiment.

FIG. 24 is a flowchart for explaining the processing flow in a first classification data generation subroutine.

FIG. 25 is a diagram for explaining the configuration of a catheter system according to a third embodiment.

FIG. 26 is a flowchart for explaining the flow of processing according to a program of the third embodiment.

FIG. 27 is an example of display according to the third embodiment.

FIG. 28 is a flowchart for explaining the flow of processing according to a program of Modification 3-1.

FIG. 29 is a diagram for explaining the configuration of a catheter system according to a fourth embodiment.

FIG. 30 is a flowchart for explaining the flow of processing according to a program of the fourth embodiment.

FIG. 31 is a functional block diagram of an information processing apparatus according to a fifth embodiment.

FIG. 32 is a functional block diagram of an image processing apparatus according to a sixth embodiment.

FIG. 33 is a functional block diagram of an image processing apparatus according to a seventh embodiment.

### DETAILED DESCRIPTION

Set forth below with reference to the accompanying drawings is a detailed description of embodiments of a learning model generation method, an image processing apparatus, an information processing apparatus, a training data generation method, and an image processing method.

### First Embodiment

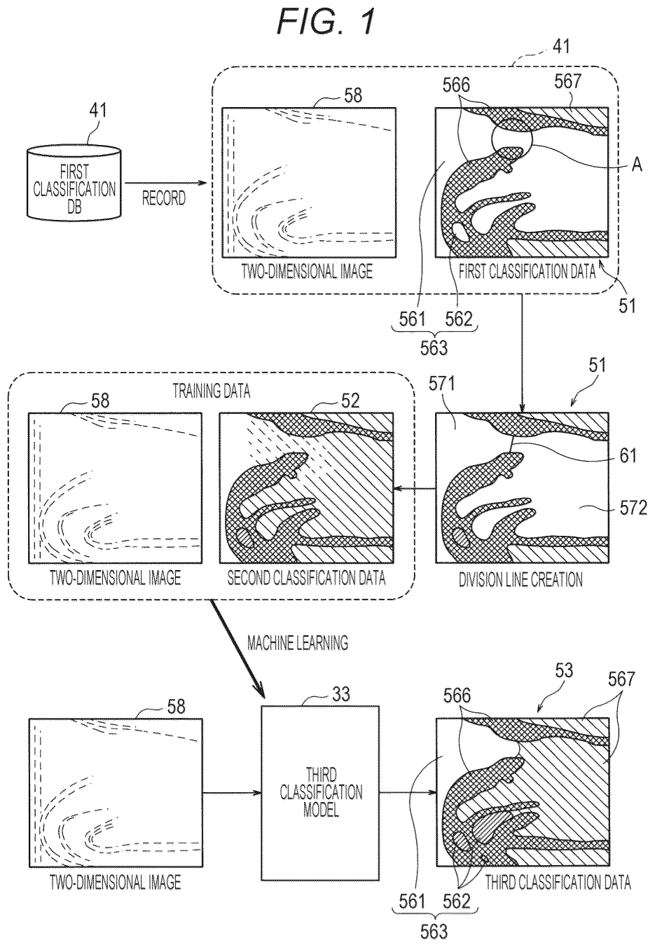

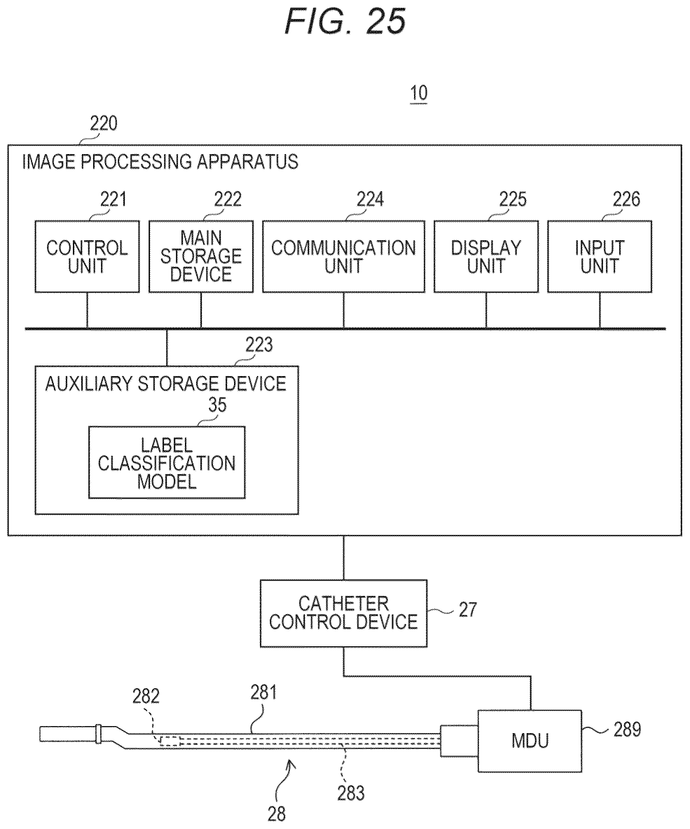

FIG. 1 is an explanatory diagram for explaining a method for generating a third classification model 33. A large number of sets of a two-dimensional image 58 and first classification data 51 are recorded in a first classification database (DB) 41. A two-dimensional image 58 of the present embodiment is a tomographic image acquired using a radial-scanning image-acquiring catheter 28 (see FIG. 25). In the description below, a case where each two-dimensional image 58 is an ultrasonic tomographic image is explained as an example.

Each two-dimensional image 58 may be a tomographic image by optical coherence tomography (OCT) using near-infrared light. The two-dimensional image 58 may be a tomographic image acquired using a linear-scanning or sector-operating image-acquiring catheter 28.

FIG. 1 illustrates a two-dimensional image 58 in a so-called R-T format formed by arranging scanning line data in parallel in the order of scanning angle. The left end of the two-dimensional image 58 represents the image-acquiring catheter 28. A horizontal direction of the two-dimensional image 58 corresponds to the distance to the image-acquiring catheter 28, and a vertical direction of the two-dimensional image 58 corresponds to the scanning angle.

The first classification data 51 is data obtained by classifying each pixel included in the two-dimensional image 58 into a living tissue region 566, a lumen region 563, and an extra-luminal region 567. The lumen region 563 is classified into a first lumen region 561 into which the image-acquiring catheter 28 is inserted, and a second lumen region 562 into which the image-acquiring catheter 28 is not inserted.

Each pixel is associated with a label indicating the region into which the pixel is classified. In FIG. 1, the portion associated with the label of the living tissue region 566 is indicated by grid hatching, the portion associated with the label of the first lumen region 561 is indicated by no hatching, the portion associated with the label of the second lumen region 562 is indicated by left-downward hatching, and the portion associated with the label of the extra-luminal region 567 is indicated by right-downward hatching. Note that a label may be associated with each small region obtained by collecting a plurality of pixels included in the two-dimensional image 58.

A case where the image-acquiring catheter 28 is inserted into a circulatory organ such as a blood vessel or the heart is now specifically described as an example. The living tissue region 566 corresponds to a luminal organ wall, such as a blood vessel wall or a heart wall. The first lumen region 561 is a region inside the luminal organ into which the image-acquiring catheter 28 is inserted. That is, the first lumen region 561 is a region filled with blood.

The second lumen region 562 is a region inside another luminal organ located in the vicinity of the blood vessel or the like into which the image-acquiring catheter 28 is inserted. For example, the second lumen region 562 is a region inside a blood vessel branched from the blood vessel into which the image-acquiring catheter 28 is inserted, or a region inside another blood vessel close to the blood vessel into which the image-acquiring catheter 28 is inserted. There also are cases where the second lumen region 562 is a region inside a luminal organ other than the circulatory organs, such as a bile duct, a pancreatic duct, a ureter, or a urethra, for example.

The extra-luminal region 567 is a region outside the living tissue region 566. When a region inside an atrium, a ventricle, a thick blood vessel, or the like is not accommodated within the display range of the two-dimensional image 58, the region is classified into the extra-luminal region 567.

Although not illustrated in the drawing, the first classification data 51 may include labels corresponding to a variety of regions such as an instrument region in which the image-acquiring catheter 28 and a guide wire or the like inserted together with the image-acquiring catheter 28 are drawn, and a lesion region in which a lesion such as calcification is drawn, for example. A method of creating the first classification data 51 from the two-dimensional image 58 will be described later.

In the first classification data 51 illustrated in FIG. 1, the first lumen region 561 is continuous from the right end to the left end of the first classification data 51. That is, since an opening exists in the living tissue region 566, the first lumen region 561 is not surrounded by the living tissue region 566. In the description below, there are cases where a state in which the first lumen region 561 is continuous from the right end to the left end of the first classification data 51 will be referred to as a state in which the first lumen region 561 is “open”. Likewise, there are cases where a state in which the first lumen region 561 is not continuous to the left end of the first classification data 51 will be described as a state in which the first lumen region 561 is “closed”.

In the example illustrated in FIG. 1, since the living tissue region 566 is not appropriately extracted in a portion A and has an opening, the first lumen region 561 is in an open state. For example, it is known that there are cases where a two-dimensional image 58 in a state where part of a living tissue is accidentally cut out or in an unclear state due to various factors such as the state of the angle between the image-acquiring catheter 28 and the inner wall of the living tissue, the distance between the image-acquiring catheter 28 and the inner wall of the living tissue, and the properties of the living tissue. In such first classification data 51 created on the basis of the two-dimensional image 58, an opening exists in part of the living tissue region 566.

In a case where the first lumen region 561 in the first classification data 51 is in an open state due to the presence of an opening in the living tissue region 566, the region outside the opening of the living tissue region 566 in the first lumen region 561 is not important information for grasping the structure of the luminal organ. Therefore, it is preferable that the first lumen region 561 does not include a region outside the opening.

For example, in a case where automatic measurement of the area, the volume, or the perimeter of each region is performed, if a region outside the opening of the living tissue region 566 is included in the first lumen region 561, there is a possibility that an error will occur in the measurement result. Further, in a case where a three-dimensional image is created using the three-dimensional scanning image-acquiring catheter 28, the region labeled with the first lumen region 561 existing outside the opening of the living tissue region 566 becomes like noise (i.e., an impediment) in the three-dimensional image in grasping the structure of the luminal organ. Therefore, it becomes rather difficult for the user to grasp the three-dimensional shape.

There are cases where a user who is not sufficiently skilled may be confused by such noise and may have difficulty in understanding the structure of the portion under observation. In a case where a user such as a skilled doctor or a medical technician views the two-dimensional image 58, it is possible to relatively easily determine that noise is generated in the three-dimensional image due to the opening of the living tissue region 566. However, to correctly perform the automatic measurement of the area and the like, for example, it may be troublesome for the user to manually correct the label of the first classification data 51.

In the present embodiment, a division line 61 that divides the first lumen region 561 into a first region 571 that is the side closer to the image-acquiring catheter 28, and a second region 572 that is the side farther from the image-acquiring catheter 28 is automatically created. The division line 61 is a line based on the assumption that there is the living tissue region 566 that divides the first lumen region 561 and the extra-luminal region 567. A specific example of a method for creating the division line 61 will be described later.

After that, for each of the pixels constituting the first lumen region 561, the probability of being the first lumen region 561 and the probability of being the extra-luminal region 567 are automatically allocated, and second classification data 52 is created. The sum of the probability of being the first lumen region 561 and the probability of being the extra-luminal region 567 is one. In the vicinity of the division line 61, the probability of being the first lumen region 561 is substantially equal to the probability of being the extra-luminal region 567. In the direction of approaching the image-acquiring catheter 28 from the division line 61, the probability of being the first lumen region 561 increases. In the direction of moving away from the image-acquiring catheter 28 from the division line 61, the probability of being the extra-luminal region 567 increases. A specific example of a probability allocation method will be described later.

For the data in which the first lumen region 561 reaches the right end of the first classification data 51 among the sets of the two-dimensional image 58 and the first classification data 51 recorded in the first classification DB 41, the second classification data 52 is created by the above process. A set of the two-dimensional image 58 and the second classification data 52 forms a set of training data.

For the data in which the first lumen region 561 does not reach the right end of the first classification data 51 among the sets of the two-dimensional image 58 and the first classification data 51 recorded in the first classification DB 41, the second classification data 52 is not created. A set of the two-dimensional image 58 and the first classification data 51 forms a set of training data.

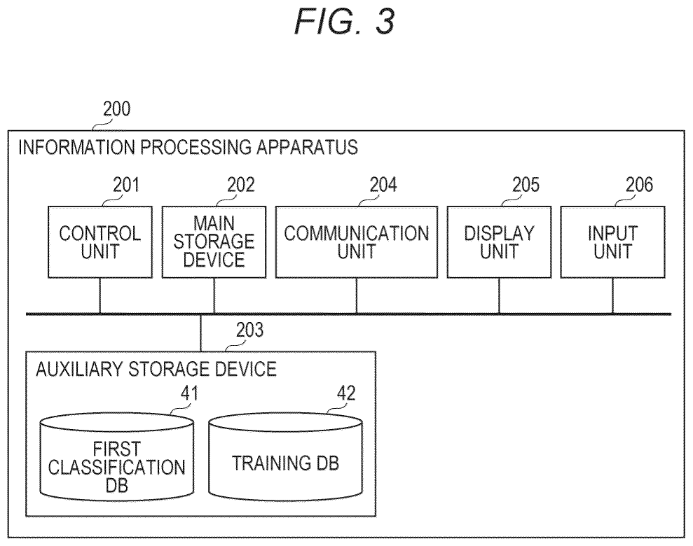

In the above manner, a training DB 42 (see FIG. 3) in which a large number of sets of training data are recorded is automatically created. Machine learning is performed using the training DB 42, and the third classification model 33 that outputs third classification data 53 is generated when the two-dimensional image 58 is input. As illustrated in FIG. 1, in the third classification data 53, a boundary line between the first lumen region 561 and the extra-luminal region 567 is created at a place where the living tissue region 566 does not exist.

In the above manner, even in a case where there is a place where a living tissue is not clearly visualized in the two-dimensional image 58, the third classification model 33 that appropriately assigns a label can be generated. The generated third classification model 33 is an example of a learning model according to the present embodiment. In the description below, the third classification model 33 for which machine learning has been completed can be called a trained model in some cases.

By outputting the third classification data 53 using the third classification model 33 generated in this manner, it is possible to provide a catheter system 10 (see FIG. 25) that aids the user in quickly understanding the structure of the portion under observation. Further, it is possible to provide the catheter system 10 that appropriately performs automatic measurement of the area and displays a three-dimensional image, without the user performing any complicated correction work.

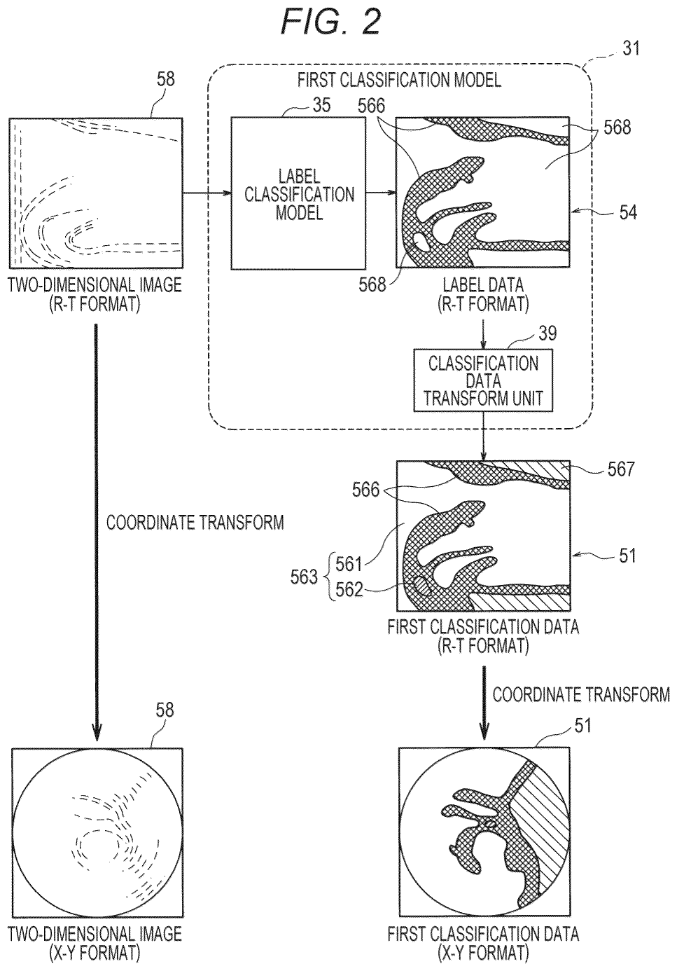

FIG. 2 is a diagram for explaining the first classification data 51. A first classification model 31 that creates the first classification data 51 on the basis of the two-dimensional image 58 includes two components: a label classification model 35 and a classification data conversion unit 39.

First, the two-dimensional image 58 is input into the label classification model 35, and label data 54 is output. The label classification model 35 can be, for example, a model that assigns, to a small region, a label related to a subject drawn in the small region such as each of the pixels constituting the two-dimensional image 58. The label classification model 35 is generated by a known machine learning technique such as semantic segmentation, for example.

In the example illustrated in FIG. 2, the label data 54 includes a label indicating the living tissue region 566 represented by grid-like hatching, and a label indicating a non-living tissue region 568 that is the other region.

The label data 54 is input to the classification data conversion unit 39, and the above-described first classification data 51 is output. Specifically, the label of the region surrounded by only the living tissue region 566 in the non-living tissue region 568 is converted into the second lumen region 562. In the non-living tissue region 568, the region in contact with the image-acquiring catheter 28, which is the left end (the center in the radial direction in the R-T format image) of the first classification data 51, is converted into the first lumen region 561.

In the non-living tissue region 568, the region that has been converted neither into the first lumen region 561 nor into the second lumen region 562, or specifically, the region whose periphery is surrounded by the living tissue region 566 and the outer end in the radial direction in the R-T format image (the right end in the label data 54 illustrated in FIG. 2) is converted into the extra-luminal region 567. Note that the upper and lower end portions of the R-T format image in the theta direction are connected, and therefore, the periphery of the extra-luminal region 567 is surrounded by the living tissue region 566 and the outer end of the R-T format image in the radial direction in the example illustrated in FIG. 2.

The two-dimensional image 58 in the R-T format and the first classification data 51 can be converted into an X-Y format by coordinate transform. Since the method of conversion between an R-T format image and an X-Y format image is known, explanation of the method of conversion is not made herein. Note that the label classification model 35 may be a model that receives the two-dimensional image 58 in the X-Y format, and outputs the label data 54 in the X-Y format. However, processing the two-dimensional image 58 in the X-Y format is not affected by an interpolation process or the like at the time of conversion from the R-T format to the X-Y format, and thus, more appropriate label data 54 is created.

The configuration of the first classification model 31 described with reference to FIG. 2 is an example. The first classification model 31 may be a model trained to receive an input of the two-dimensional image 58, and directly output the first classification data 51.

The label classification model 35 is not necessarily a model using machine learning. The label classification model 35 may be a model that extracts the living tissue region 566 on the basis of a known image processing method, for example, such as edge extraction.

Instead of the first classification model 31, an expert skilled in interpretation of the two-dimensional image 58 may paint the two-dimensional image 58 in each region, to create the first classification data 51. The set of the two-dimensional image 58 and the first classification data 51 created in this manner can be used as training data when the first classification model 31 or the label classification model 35 is generated by machine learning.

FIG. 3 is a diagram for explaining the configuration of an information processing apparatus 200 that creates a training database (DB). The information processing apparatus 200 can include a control unit 201, a main storage device 202, an auxiliary storage device 203, a communication unit 204, a display unit 205, an input unit 206, and a bus. The control unit 201 is an arithmetic control device that executes a program according to the present embodiment. For the control unit 201, one or a plurality of central processing units (CPUs) or graphics processing units (GPUs), a multi-core CPU, or the like, can be used. The control unit 201 is connected to each of the hardware components constituting the information processing apparatus 200 via the bus.

The main storage device 202 is a storage device such as a static random access memory (SRAM), a dynamic random access memory (DRAM), or a flash memory. The main storage device 202 temporarily stores the information necessary in the middle of processing being performed by the control unit 201, and the program being executed by the control unit 201.

The auxiliary storage device 203 is a storage device such as an SRAM, a flash memory, a hard disk, or a magnetic tape. The auxiliary storage device 203 stores the first classification database (DB) 41, the training DB 42, the program to be executed by the control unit 201, and various kinds of data necessary in executing the program. The communication unit 204 is an interface that conducts communication between the information processing apparatus 200 and a network. The first classification DB 41 and the training DB 42 may be stored in an external mass storage device or the like connected to the information processing apparatus 200.

The display unit 205 can be, for example, a liquid crystal display panel, an organic electro-luminescence (EL) panel, or the like. The input unit 206 can be, for example, a keyboard, a mouse, or the like. The input unit 206 may be stacked on the display unit 205, to form a touch panel. The display unit 205 may be a display device connected to the information processing apparatus 200. The information processing apparatus 200 may not include the display unit 205 and the input unit 206.

The information processing apparatus 200 can be, for example, a general-purpose personal computer, a tablet, a large computing machine, or a virtual machine that runs on a large computing machine. The information processing apparatus 200 may be formed with a plurality of personal computers that perform distributed processing, or hardware such as a large computing machine. The information processing apparatus 200 may be formed with a cloud computing system or a quantum computer.

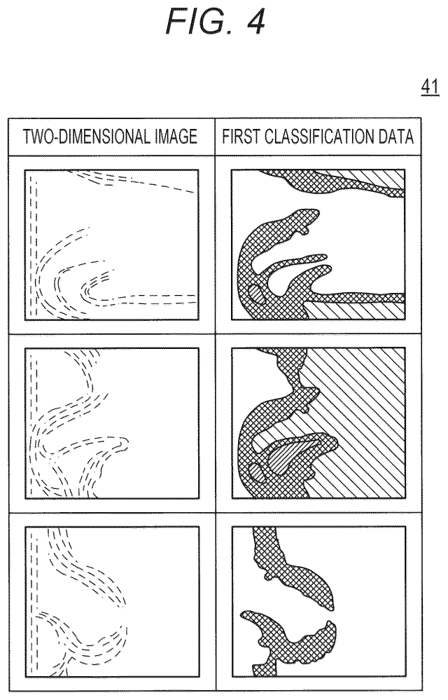

FIG. 4 is a diagram for explaining the record layout in the first classification DB 41. The first classification DB 41 is a database (DB) that records the two-dimensional images 58 and the first classification data 51 that are associated with each other. The first classification DB 41 has a two-dimensional image field and a first classification data field. The two-dimensional images 58 are recorded in the two-dimensional image field. The first classification data 51 is recorded in the first classification data field.

The first classification DB 41 records a large number of sets of the two-dimensional images 58 collected from many medical institutions and the first classification data 51 created by the method described above with reference to FIG. 2, for example, on the basis of the two-dimensional images 58. The first classification DB 41 has one record for one two-dimensional image 58.

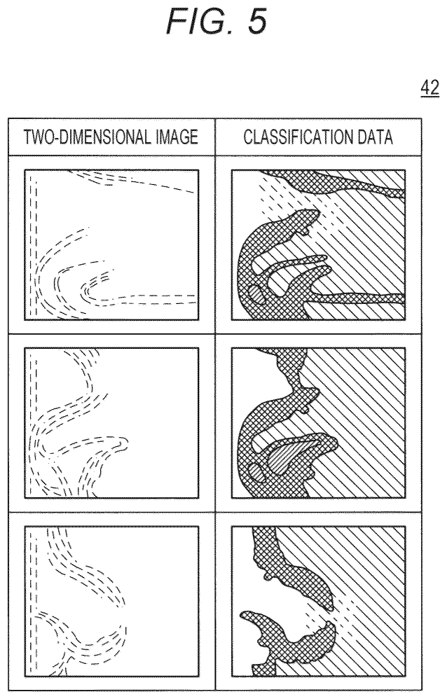

FIG. 5 is a diagram for explaining the record layout in the training DB 42. The training DB 42 is a database (DB) that records the two-dimensional images 58 and classification data that are associated with each other. The training DB 42 has a two-dimensional image field and a classification data field. The two-dimensional images 58 are recorded in the two-dimensional image field. Classification data associated with the two-dimensional images 58 is recorded in the classification data field.

The two-dimensional images 58 recorded in the two-dimensional image field of the training DB 42 are the same as the two-dimensional images 58 recorded in the two-dimensional image field of the first classification DB 41. The classification data recorded in the classification data field of the training DB 42 is the first classification data 51 recorded in the first classification data field of the first classification DB 41 or the second classification data 52 created on the basis of the first classification data 51. The training DB 42 has one record for one two-dimensional image 58.

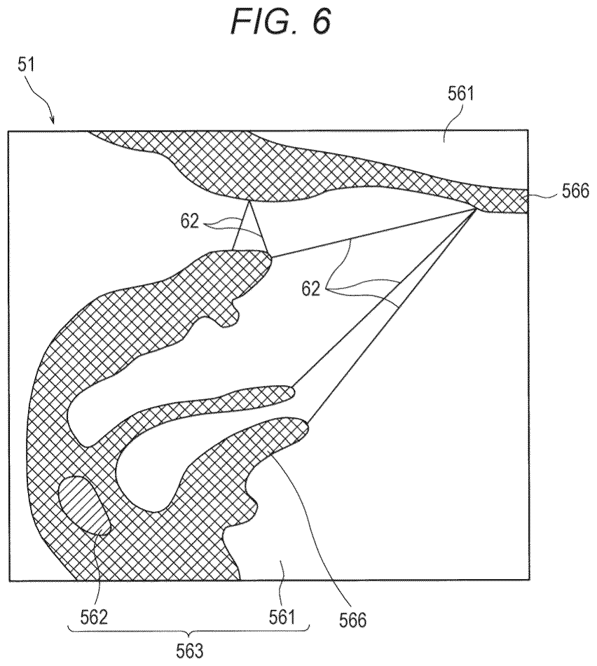

FIG. 6 is a diagram for explaining a method for creating the division line 61. FIG. 6 illustrates the first classification data 51 in which the first lumen region 561 is in an open state. The living tissue region 566 is separately illustrated in two portions on the upper side and the lower side.

In FIG. 6, five candidate division lines 62 are created between the living tissue region 566 on the upper side and the living tissue region 566 on the lower side. The candidate division lines 62 can be created at any appropriate positions, as long as the upper and lower living tissue regions 566 are connected. For example, the control unit 201 selects a first point at a random position in the living tissue region 566 on the upper side, and selects a second point at a random position in the living tissue region 566 on the lower side. The control unit 201 determines that the portions interposed between the upper living tissue region 566 and the lower living tissue region 566 among the straight lines connecting the first point and the second point are the candidate division lines 62.

After that, the first classification data 51 selects one division line 61 from the plurality of candidate division lines 62. For example, the control unit 201 selects the shortest candidate division line 62 as the division line 61 among the plurality of candidate division lines 62. The control unit 201 may randomly select one candidate division line 62 as the division line 61 from among the plurality of candidate division lines 62. Modifications of the method for determining the division line 61 will be described later.

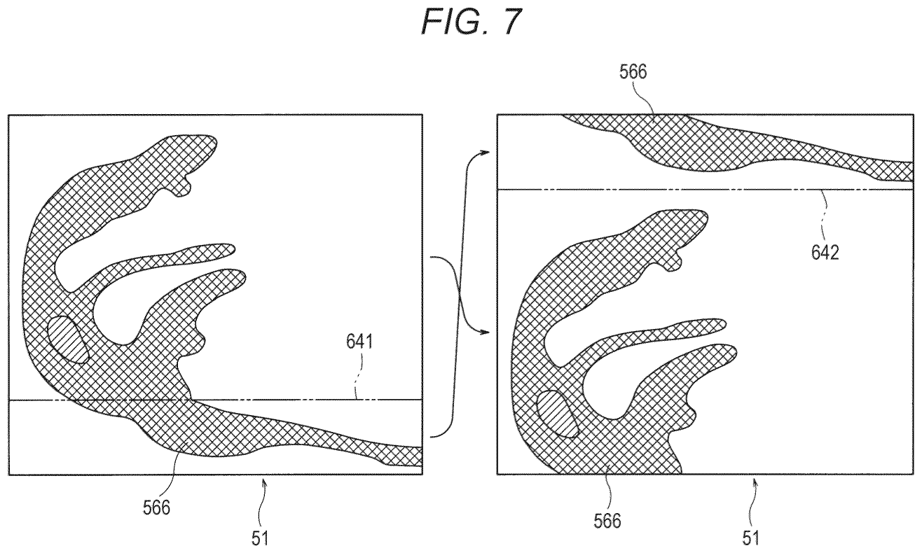

FIG. 7 is a diagram for explaining a process to be performed in a case where an opening of the living tissue region 566 is present at the end of an R-T format image in the theta direction (the end of the first classification data 51 in the vertical direction as illustrated in FIG. 7). The left side of FIG. 7 illustrates an example of an R-T image in a case where the scanning angle at which the display of the R-T format image is started matches the direction in which the living tissue region 566 looks open. The living tissue region 566 is drawn in a single mass (i.e., a single non-separated region), and is not in contact with the upper and lower edges of the R-T format image. In such a state, it is difficult to create the candidate division lines 62.

The control unit 201 can convert such an R-T format image into an R-T image as illustrated on the right side of FIG. 7 by cutting the R-T format image along a cutting line 641 parallel to the scanning line, and attaching the lower cut portion to the upper edge of the R-T format image along an attachment line 642. By making the openings of the living tissue region 566 face each other, the control unit 201 can create the candidate division lines 62, using the procedures described above with reference to FIG. 6.

The control unit 201 can obtain a two-dimensional image 58 in which the candidate division lines 62 can be created, by similar procedures that include changing the scanning angle at which the display of the R-T format image is started, instead of cutting and attaching the R-T format image.

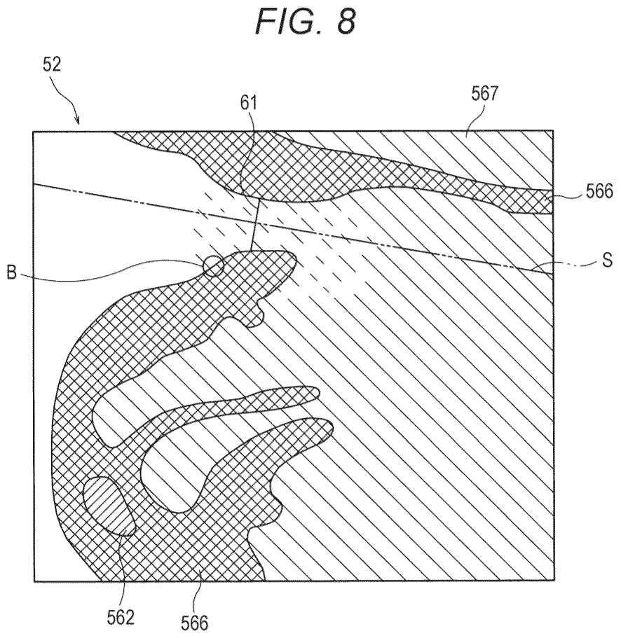

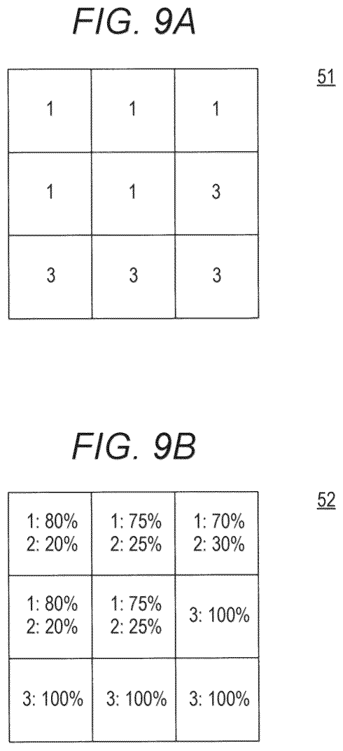

FIGS. 8 to 12 are diagrams for explaining the second classification data 52. FIG. 9A is a schematic diagram illustrating, in an enlarged manner, nine pixels at the place corresponding to a portion B in FIG. 8 in the first classification data 51. Each pixel is associated with a label, for example, such as “1”, “2” or “3”. In the description below, “1” is the label indicating the first lumen region 561, “2” is the label indicating the extra-luminal region 567, and “3” is the label indicating the living tissue region 566.

FIG. 9B is an enlarged schematic diagram illustrating the nine pixels in the portion B in FIG. 8. FIGS. 9A and 9B illustrate the pixels at the same position. In FIG. 9B, the label “1: 80%, 2: 20%” associated with the upper left pixel indicates that “the probability of being the first lumen region 561 is 80%, and the probability of being the extra-luminal region 567 is 20%”. In any pixel, the probability of being the first lumen region 561 and the probability of being the extra-luminal region 567 are allocated so that the sum of the probabilities is 100%.

Likewise, the label “3: 100%” associated with the lower right pixel indicates that “the probability of being the living tissue region 566 is 100%”. The pixel associated with the label “3” in FIG. 9A is associated with the label “3: 100%” in FIG. 9B. In this manner, in the second classification data 52, the probabilities of a plurality of labels can be associated with one pixel.

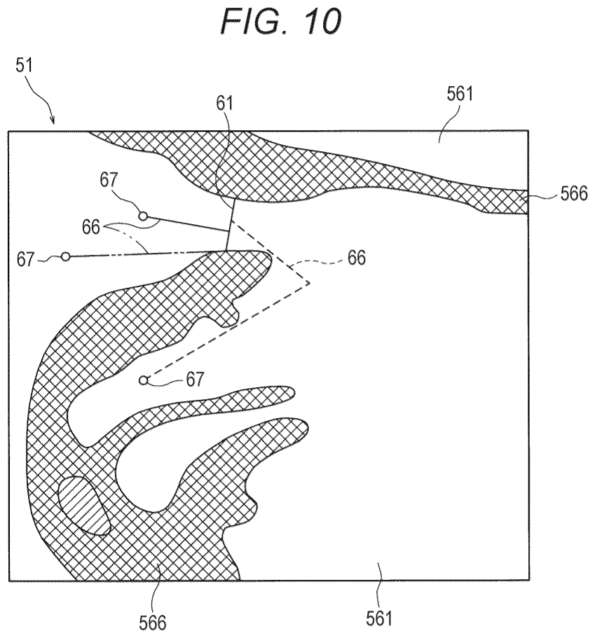

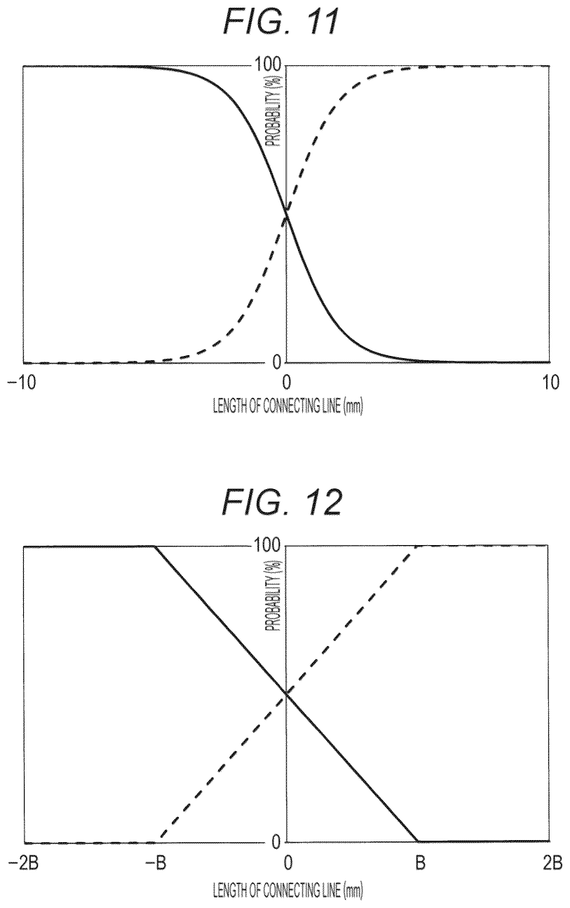

Referring now to FIGS. 10 and 11, An example of a method of determining the probability corresponding to each pixel is described. FIG. 10 schematically illustrates three target pixels 67 and connecting lines 66 corresponding to the respective target pixels 67. The connecting lines 66 are lines connecting the target pixels 67 and the division line 61.

A solid connecting line 66 indicates an example of a connecting line 66 drawn perpendicularly from a target pixel 67 toward the division line 61. A two-dot-and-dash connecting line 66 indicates an example of a connecting line 66 drawn obliquely from a target pixel 67 toward the division line 61. A dashed connecting line 66 indicates an example of a connecting line 66 that is drawn from a target pixel 67 toward the connecting line 66 and is bent once.

The control unit 201 sequentially determines the respective pixels constituting the first lumen region 561 as the target pixels 67, creates the connecting lines 66 so as not to intersect the living tissue region 566, and calculates the lengths of the connecting lines 66. The perpendicular connecting line 66 indicated by the solid line has the highest priority in creating the connecting lines 66. In a case where a connecting line 66 perpendicular to the division line 61 cannot be created from any target pixel 67, the control unit 201 creates a connecting line 66 so as to be the shortest straight line that does not intersect the living tissue region 566, as indicated by the two-dot-and-chain line, and calculates the length of the connecting line 66 that is the shortest straight line that does not intersect the living tissue region 566.

In a case where a connecting line 66 connecting a target pixel 67 and the division line 61 with a straight line cannot be created, the control unit 201 creates a connecting line 66 so as not to intersect the living tissue region 566 as indicated by the dashed line and to be the shortest bent line, and calculates the length of the connecting line 66 that is the shortest bent line. In a case where a connecting line 66 cannot be created with a line that is bent once, the control unit 201 creates a connecting line 66 with a line that is bent twice or more.

FIG. 11 is an example of a graph showing the relationship between the lengths of the connecting lines 66, and the probability of being the first lumen region 561 and the probability of being the extra-luminal region 567. The abscissa axis indicates the lengths of the connecting lines 66. On the abscissa axis, “0” indicates that it is on the division line 61. The positive direction of the abscissa axis indicates the lengths of the connecting lines 66 belonging to the right side of the division line 61, which is the region on the side farther from the image-acquiring catheter 28. The negative direction of the abscissa axis indicates the lengths of the connecting lines 66 belonging to the left side of the division line 61, which is the region on the side closer to the image-acquiring catheter 28.

For example, the probability of being the first lumen region 561 and the probability of being the extra-luminal region 567 on an imaginary line S drawn perpendicular to the division line 61 in FIG. 8 are indicated by the graph shown in FIG. 11. Here, the origin of the abscissa axis corresponds to the intersection of the division line 61 and the imaginary line S.

The ordinate axis in FIG. 11 indicates probability. A solid line indicates the probability of being the first lumen region 561 in percentage terms. A dashed line indicates the probability of being the extra-luminal region 567 in percentage terms. The probability illustrated in FIG. 11 is a sigmoid curve shown in Expressions (1) to (4)

Math. 1

where the target pixel 67 is closer to the image-acquiring catheter 28 than the division line 61

$$P 1 = \frac{1}{1 + {e}^{- \frac{L}{A}}} \qquad ( 1 )$$

$$P 2 = 1 - P 1 \qquad ( 2 )$$

where the target pixel 67 is farther from the image-acquiring catheter 28 than the division line 61

$$P 1 = \frac{1}{1 + {e}^{\frac{L}{A}}} \qquad ( 3 )$$

$$P 2 = 1 - P 1 \qquad ( 4 )$$

P1: probability that the small regions are in a lumen region;

P2: probability that the small regions are in an extra-luminal region;

L: length of the connecting line; and

A: constant.

Note that FIG. 11 illustrates an example of the graph in a case where a constant A=1.

FIG. 12 is a modification of the graph showing the relationship between the lengths of the connecting lines 66, and the probability of being the first lumen region 561 and the probability of being the extra-luminal region 567. The ordinate axis and the abscissa axis, and the meanings of the solid line graph and the dashed line graph are similar to those in FIG. 11, and therefore, explanation of them is not made herein. B shown on the abscissa axis is a constant.

In FIG. 12, in a case where the length of the connecting line 66 is smaller than “−B”, which is a case where the connecting line is closer to the image-acquiring catheter 28 than the threshold value B, the probability that the connecting line is in the first lumen region 561 is 100%. Likewise, in a case where the length of the connecting line 66 is greater than “+B”, which is a case where the connecting line is farther from the image-acquiring catheter 28 than the threshold value B, the probability that the connecting line is in the extra-luminal region 567 is 100%. In a range where the length of the connecting line 66 is from “−B” to “+B”, the probability of being the first lumen region 561 monotonously decreases linearly, and the probability of being the extra-luminal region 567 monotonously increases linearly.

The probability of being the first lumen region 561 and the probability of being the extra-luminal region 567 are not necessarily as illustrated by the graphs shown in FIGS. 11 and 12. The parameters A and B can be selected as appropriate. For example, the probability of being the first region 571 may be 100% on the right side of the division line 61, and the probability of being the extra-luminal region 567 may be 100% on the left side of the division line 61.

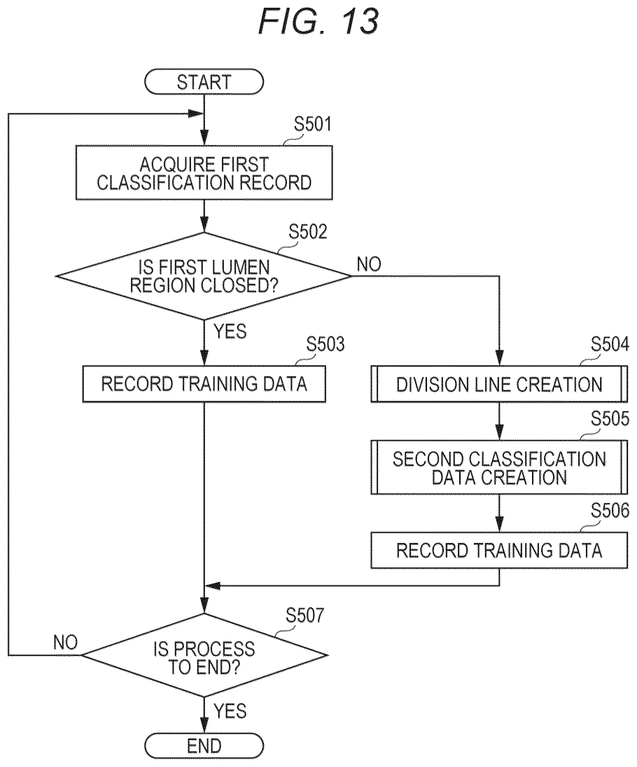

FIG. 13 is a flowchart illustrating a flow of processing according to a program. The control unit 201 acquires one set of first classification records from the first classification DB 41 (S501). Through S501, the control unit 201 achieves the functions of an image acquisition unit and the functions of a first classification data acquisition unit of the present embodiment.

The control unit 201 determines whether the first lumen region 561 is in a closed state (S502). Through S502, the control unit 201 achieves the functions of a determination unit of the present embodiment. If the first lumen region 561 is determined to be in a closed state (YES in S502), the control unit 201 creates a new record in the training DB 42, and records the two-dimensional image 58 and the first classification data 51 recorded in the record acquired in S501 (S503).

If the first lumen region 561 is determined not to be in a closed state (NO in S502), the control unit 201 starts a division line creation subroutine (S504). The division line creation subroutine is a subroutine for creating the division line 61 that divides the first lumen region 561 in an open state into the first region 571 on the side closer to the image-acquiring catheter 28 and the second region 572 on the side farther from the image-acquiring catheter 28. Through the division line creation subroutine, the control unit 201 achieves the functions of a division line creation unit of the present embodiment. The flow of processing of the division line creation subroutine will be described later.

The control unit 201 starts a second classification data creation subroutine (S505). The second classification data creation subroutine is a subroutine for creating the second classification data 52 in which the probability of being the first lumen region 561 and the probability of being the extra-luminal region 567 are allocated to each of the small regions constituting the first lumen region 561 of the first classification data 51. Through the second classification data creation subroutine, the control unit 201 achieves the functions of a second classification data generation unit of the present embodiment. The flow of processing in the second classification data creation subroutine will be described later.

The control unit 201 creates a new record in the training DB 42, and records a two-dimensional image 58 and the second classification data 52 (S506). Here, the two-dimensional image 58 is the two-dimensional image 58 recorded in the record acquired in S501. The second classification data 52 is the second classification data 52 created in S505.

After S503 or S506 is completed, the control unit 201 determines whether to end the processing (S507). For example, in a case where the processing of all the records recorded in the first classification DB 41 has been completed, the control unit 201 determines to end the processing. The control unit 201 may determine to end the processing in a case where the processing of a predetermined number of records has been completed.

If the control unit 201 determines not to end the processing (NO in S507), the control unit 201 returns to S501. If the control unit 201 determines to end the processing (YES in S507), the control unit 201 ends the processing.

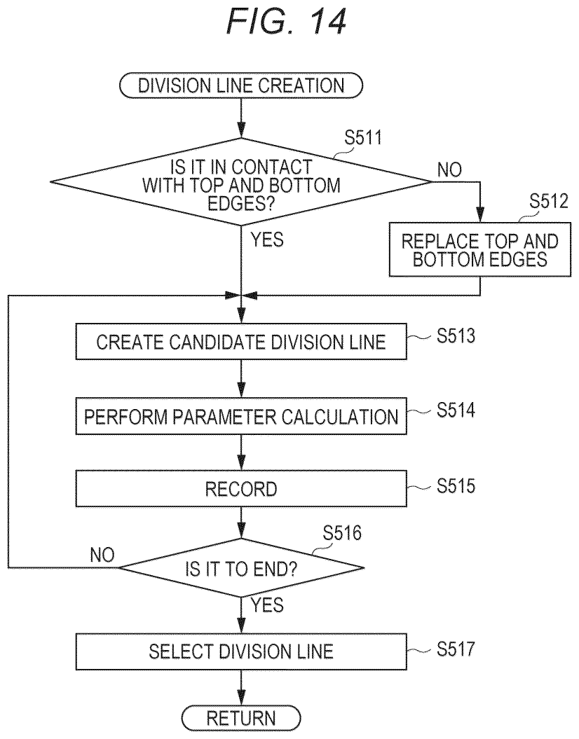

FIG. 14 is a flowchart for explaining the processing flow in the division line creation subroutine. The division line creation subroutine is a subroutine for creating the division line 61 that divides the first lumen region 561 in an open state into the first region 571 on the side closer to the image-acquiring catheter 28 and the second region 572 on the side farther from the image-acquiring catheter 28.

The control unit 201 determines whether the living tissue region 566 included in the first classification data 51 is in contact with upper and lower edges of the R-T format image (S511). If the control unit 201 determines that the living tissue region 566 is not in contact with the upper and lower edges (NO in S511), the control unit 201 cuts the first classification data 51 along the cutting line 641 extending through the living tissue region 566 as described with reference to FIG. 7, and attaches the lower cut portion to the upper edge (S512).

If the control unit 201 determines that the living tissue region 566 is in contact with the upper and lower edge (YES in S511), or after the end of S512, the control unit 201 creates one divided candidate division line 62 (S513). A specific example is now described. The control unit 201 selects the first point at a random position in the living tissue region 566 on the upper side. The control unit 201 selects the second point at a random position in the living tissue region 566 on the lower side. The control unit 201 determines that the portions interposed between the upper living tissue region 566 and the lower living tissue region 566 among the straight lines connecting the first point and the second point are the candidate division lines 62.

The control unit 201 may create a candidate division line 62 so as to cover the combinations of the respective pixels in the living tissue region 566 on the upper side and the respective pixels in the living tissue region 566 on the lower side.

The control unit 201 calculates a predetermined parameter related to the candidate division line 62 (S514). The parameter is the length of the candidate division line 62, the area of a region that is closer to the image-acquiring catheter 28 than the candidate division line 62 in the first lumen region 561, the inclination of the candidate division line 62, or the like.

The control unit 201 associates the start point and the end point of the candidate division line 62 with the calculated parameter, and temporarily records the start and end points and the parameter in the main storage device 202 or the auxiliary storage device 203 (S515). Table 1 shows an example of the data to be recorded in S515 in a tabular format.

TABLE 1

| | Start point | | End point | | |
|---|---|---|---|---|---|
| | R-coordinate | T-coordinate | R-coordinate | T-coordinate | Param- |
| No. | [mm] | [degree] | [mm] | [degree] | eter |
| 1 | 15 | 320 | 16 | 215 | *** |
| 2 | 29 | 345 | 12 | 222 | *** |

The control unit 201 determines whether to end the processing (S516). For example, in a case where a predetermined number of candidate division lines 62 have been created, the control unit 201 determines to end the processing. The control unit 201 may determine to end the processing in a case where the parameter calculated in S514 satisfies a predetermined condition.

If the control unit 201 determines not to end the processing (NO in S516), the control unit 21 returns to S513. If the control unit 201 determines to end the processing (YES in S516), the control unit 201 selects the division line 61 from among the candidate division lines 62 recorded in S515 (S517). After that, the control unit 201 ends the processing.

For example, the control unit 201 calculates the lengths of the candidate division lines 62 in S514, and selects the shortest candidate division line 62 in S517. The control unit 201 may calculate the inclinations of the candidate division lines 62 in S514, and select the candidate division line 62 whose angle with the R axis is the closest to the right angle in S517. The control unit 201 may calculate a plurality of parameters in S514, and select the division line 61 on the basis of the result of the calculation.

Note that, in S517, the user may select the division line 61 from the plurality of candidate division lines 62. Specifically, the control unit 201 superimposes the plurality of candidate division lines 62 on the two-dimensional image 58 or the first classification data 51, and outputs the superimposed data to the display unit 205. The user operates the input unit 206, to select the candidate division line 62 the user has determined to be appropriate. The control unit 201 determines the division line 61 on the basis of the selection made by the user.

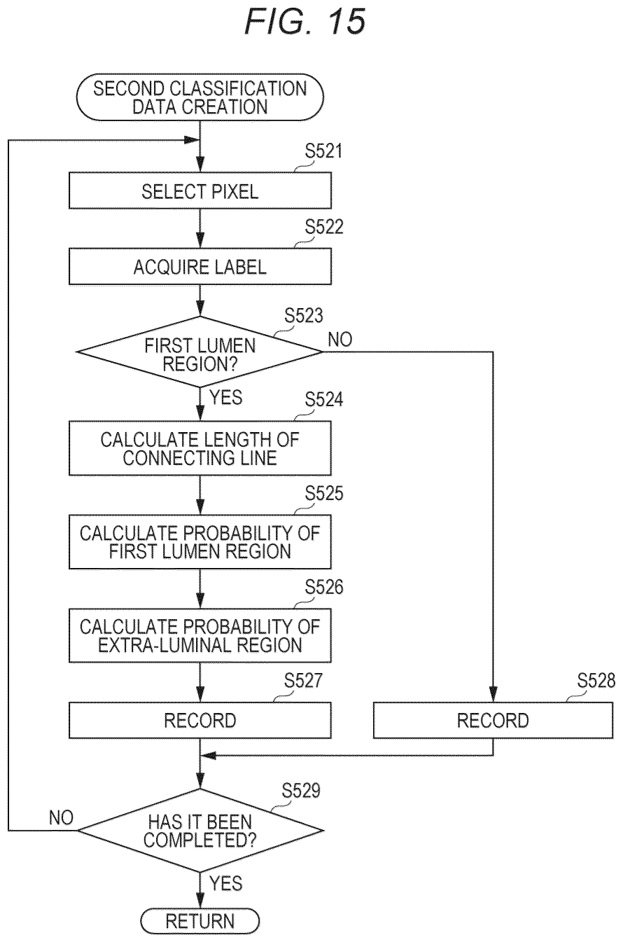

FIG. 15 is a flowchart for explaining the processing flow in the second classification data creation subroutine. The second classification data creation subroutine is a subroutine for creating the second classification data 52 in which the probability of being the first lumen region 561 and the probability of being the extra-luminal region 567 are allocated to each of the small regions constituting the first lumen region 561 of the first classification data 51.

The control unit 201 selects one of the pixels constituting first classification data 51 (S521). The control unit 201 acquires the label associated with the selected pixel (S522). The control unit 201 determines whether the label corresponds to the first lumen region 561 (S523).

If the label is determined to correspond to the first lumen region 561 (YES in S523), the control unit 201 calculates the length of the connecting line 66 that connects the pixel selected in S521 and the division line 61 without passing through the living tissue region 566 (S524). For example, the control unit 201 calculates the probability that the pixel selected in S521 is in the first lumen region 561, on the basis of the relationship between the length of the connecting line 66 and the probability described with reference to FIG. 11 or FIG. 12 (S525). Likewise, the control unit 201 calculates the probability that the pixel selected in S521 is in the extra-luminal region 567 (S526).

As described above with reference to FIG. 9B, the control unit 201 associates the position of the pixel selected in S521 with the probability calculated in each of S525 and S526, and records the position and the probability in the second classification data 52 (S527). Through S527, the control unit 201 achieves the functions of a second recording unit of the present embodiment.

If it is determined that the label does not correspond to the first lumen region 561 (NO in S523), the control unit 201 associates the position of the pixel connected in S521 with the fact that the probability of being the label acquired in S522 is 100%, and records the position and the probability in the second classification data 52 (S528). Through S528, the control unit 201 achieves the functions of a first recording unit of the present embodiment.

The control unit 201 determines whether the processing of all the pixels of the first classification data 51 has been completed (S529). When it is determined that the processing has not been completed (NO in S529), the control unit 201 returns to S521. If it is determined that the processing has been completed (YES in S529), the control unit 201 ends the processing.

Note that, in S521, the control unit 201 may select a small region formed with a plurality of pixels, and thereafter, perform processing for each small region. In a case where processing is formed for each small region, the control unit 201 performs processing of the entire small region on the basis of the label associated with the pixel at a specific position in the small region, for example.

As described above, the control unit 201 executes the program and the subroutines described with reference to FIGS. 13 to 15, and creates the training DB 42 on the basis of the first classification DB 41. For example, the training DB 42 created by each institution of a plurality of medical institutions or the like may be integrated into one database to create a large-scale training DB 42.

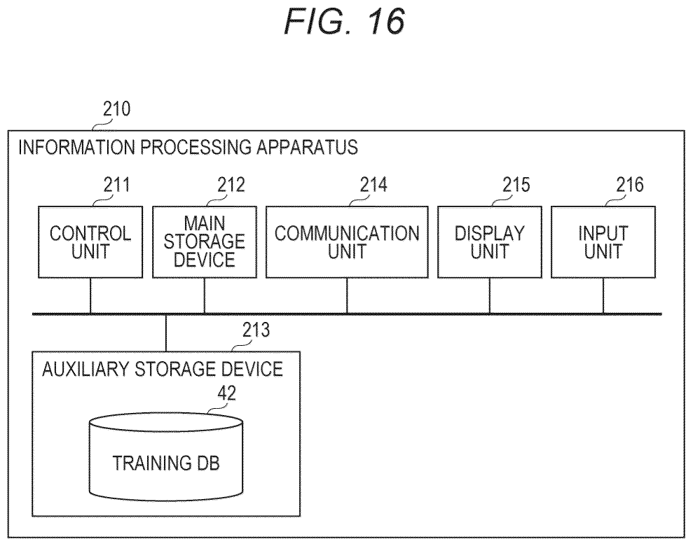

Next, a process of generating the third classification model 33 on the basis of the created training DB 42 is described. FIG. 16 is a diagram for explaining the configuration of an information processing apparatus 210 that creates a third classification model.

The information processing apparatus 210 can include a control unit 211, a main storage device 212, an auxiliary storage device 213, a communication unit 214, a display unit 215, an input unit 216, and a bus. The control unit 211 is an arithmetic control device that executes a program according to the present embodiment. For the control unit 211, one or a plurality of CPUs or GPUs, a multi-core CPU, a tensor processing unit (TPU), or the like is used. The control unit 211 is connected to each of the hardware components constituting the information processing apparatus 210 via the bus.

The main storage device 212 is a storage device such as an SRAM, a DRAM, or a flash memory. The main storage device 212 temporarily stores the information necessary in the middle of processing being performed by the control unit 211, and the program being executed by the control unit 211.

The auxiliary storage device 213 is a storage device such as an SRAM, a flash memory, a hard disk, or a magnetic tape. The auxiliary storage device 213 stores the training DB 42, the program to be executed by the control unit 211, and various kinds of data necessary for executing the program. The training DB 42 may be stored in an external mass storage device or the like connected to the information processing apparatus 210.

The communication unit 214 is an interface that conducts communication between the information processing apparatus 210 and a network. For example, the display unit 215 is a liquid crystal display panel, an organic EL panel, or the like. The input unit 216 can be, for example, a keyboard, a mouse, or the like.

The information processing apparatus 210 can be, for example, a general-purpose personal computer, a tablet, a large computing machine, a virtual machine that runs on a large computing machine, or a quantum computer. The information processing apparatus 210 may be formed with a plurality of personal computers that perform distributed processing, or hardware such as a large computing machine.

The information processing apparatus 210 may be formed with a cloud computing system or a quantum computer.

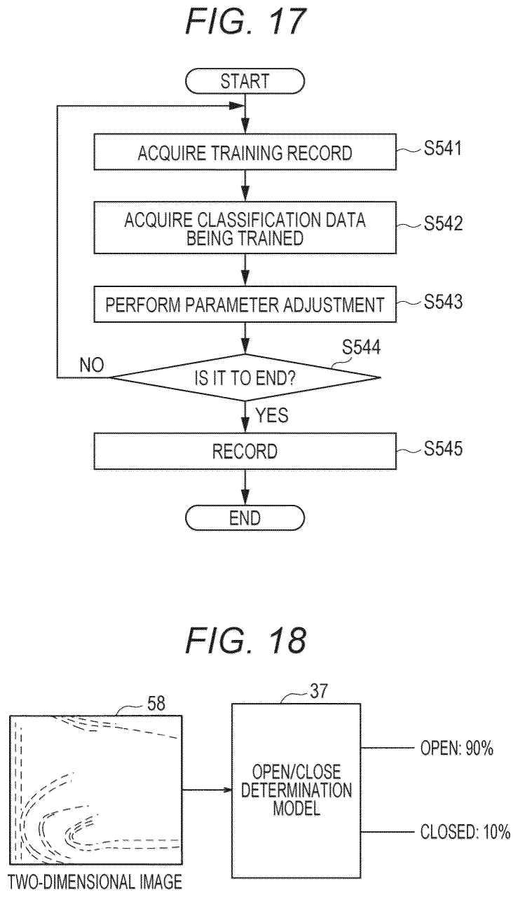

FIG. 17 is a flowchart for explaining a flow of processing according to a program for machine learning. Prior to execution of the program illustrated in FIG. 17, an untrained model, for example, such as a U-Net structure that realizes semantic segmentation is prepared. The U-Net structure includes multiple encoder layers, and multiple decoder layers connected behind the encoder layers. Each encoder layer includes a pooling layer and a convolution layer. Through semantic segmentation, a label is given to each of the pixels constituting an input image. Note that the untrained model may be a mask region-based convolutional neural network (Mask R-CNN) model, or any other model that realizes image segmentation.

For example, the label classification model 35 described with reference to FIG. 2 may be used for an untrained third classification model 33. By transfer learning in which learning for outputting the third classification data 53 is additionally performed on the label classification model 35 for which learning for outputting the label data 54 has been completed, machine learning of the third classification model 33 can be realized with less training data and a fewer number of times of learning.

The control unit 211 acquires a training record from the training DB 42 (S541). The control unit 211 inputs the two-dimensional image 58 included in the acquired training record into the third classification model 33 being trained, and acquires output data. In the description below, the data to be output from the third classification model 33 being trained will be referred to as the classification data being trained. The third classification model 33 being trained is an example of a learning model being trained according to the present embodiment.

The control unit 211 adjusts the parameters of the third classification model 33 so as to reduce the difference between the second classification data 52 included in the training record acquired in S541 and the classification data being trained (S543). Here, the difference between the second classification data 52 and the classification data being trained is evaluated on the basis of the number of pixels having different labels, for example. For adjusting the parameters of the third classification model 33, a known machine learning technique, for example, such as stochastic gradient descent (SGD) or adaptive moment estimation (Adam) can be used.

The control unit 211 determines whether to end the parameter adjustment (S544). For example, in a case where learning is repeated the predetermined number of times defined by a hyperparameter, the control unit 211 determines to end the processing. The control unit 211 may acquire test data from the training DB 42, input the test data to the third classification model 33 being trained, and determine to end the processing when an output with predetermined accuracy is obtained.

If the control unit 211 determines not to end the processing (NO in S544), the control unit 211 returns to S541. If the control unit 211 determines to end the processing (YES in S544), the control unit 211 records the adjusted parameters in the auxiliary storage device 213 (S545). After that, the control unit 211 ends the processing. Thus, the training of the third classification model 33 is completed.

According to the present embodiment, it is possible to provide the third classification model 33 that distinguishes and classifies the first lumen region 561 into which the image-acquiring catheter 28 is inserted and the extra-luminal region 567 outside the living tissue region 566, even in a case where a two-dimensional image 58 drawn in a state where part of the living tissue region 566 forming a luminal organ is missing is input. By displaying the third classification data 53 classified using the third classification model 33, it is possible to aid the user in quickly understanding the structure of the luminal organ.

By classifying the two-dimensional image 58 using the third classification data 53, it is possible to appropriately perform automatic measurement of the cross-sectional area, the volume, and the perimeter of the first lumen region 561, for example.

By classifying the two-dimensional images 58 acquired in time series with the image-acquiring catheter 28 for three-dimensional scanning using the third classification model 33, it is possible to generate a three-dimensional image with less noise.

### Modification 1-1

In the present modification, an open/close determination model 37 generated using machine learning is used in determining whether the first lumen region 561 is in a closed state. Explanation of the same portions as those of the first embodiment is not made herein. FIG. 18 is a diagram for explaining the open/close determination model.

The open/close determination model 37 receives an input of a two-dimensional image 58, and outputs the probability that the first lumen region 561 is in an open state and the probability that the first lumen region 561 is in a closed state. In FIG. 18, information indicating that the probability of being in an open state is 90% and the probability of being in a closed state is 10% is output.

The open/close determination model 37 is generated by machine learning using a large number of sets of training data in which the two-dimensional images 58 are associate with information indicating whether the first lumen region 561 is in an open state or a closed state. In S502 described with reference to FIG. 13, the control unit 201 inputs a two-dimensional image 58 to the open/close determination model 37. In a case where the probability of being in an open state exceeds a predetermined threshold, the control unit 201 determines that the first lumen region 561 is in an open state (YES in S502). The open/close determination model 37 is an example of a reach determination model according to the present embodiment.

### Modification 1-2

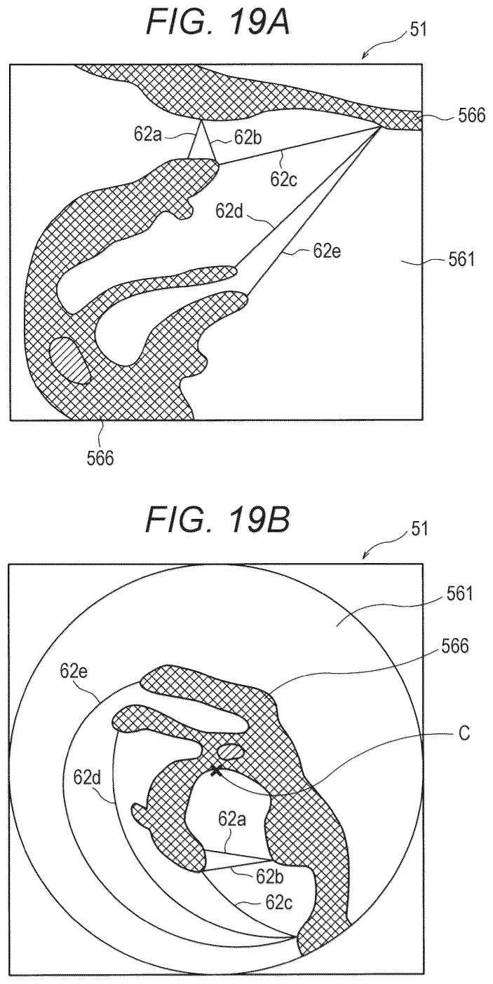

In the present modification, both an R-T format image and an X-Y format image are used in selecting the division line 61 from a plurality of candidate division lines 62. Explanation of the same portions as those of the first embodiment is not made herein. FIG. 19 is a diagram for explaining a method for selecting the division line 61 according to Modification 1-2. FIG. 19A is a diagram for explaining a state in which a plurality of candidate division lines 62 is created for the first classification data 51 displayed in an R-T format. Five candidate division lines 62 from a candidate division line 62a to a candidate division line 62e are created between the living tissue region 566 on the upper side and the living tissue region 566 on the lower side. Each of the candidate division lines 62 is a straight line. Note that the candidate division lines 62 illustrated in FIG. 19 are an example for ease of explanation.

FIG. 19B is a diagram for explaining a state in which FIG. 19A is coordinate-transformed into an X-Y format. The center C indicates the center of the first classification data 51, which is the central axis of the image-acquiring catheter 28. By coordinate transform, the candidate division lines 62a to 62e are transformed into substantially arc shapes.

In FIG. 19B, in a case where both ends of the candidate division line 62d and the candidate division line 62e are connected by straight lines, these lines intersect the living tissue region 566. In the present modification, any of the candidate division lines 62 that intersect the living tissue region 566 in a case where coordinate transform into the X-Y format has been performed is not selected as the division line 61. The candidate division lines 62a to 62c do not intersect the living tissue region 566 in a case where both ends are connected by a straight line. Any of these candidate division lines 62 might be selected as the division line 61. The parameter related to each candidate division line 62 may be determined on the X-Y format image.

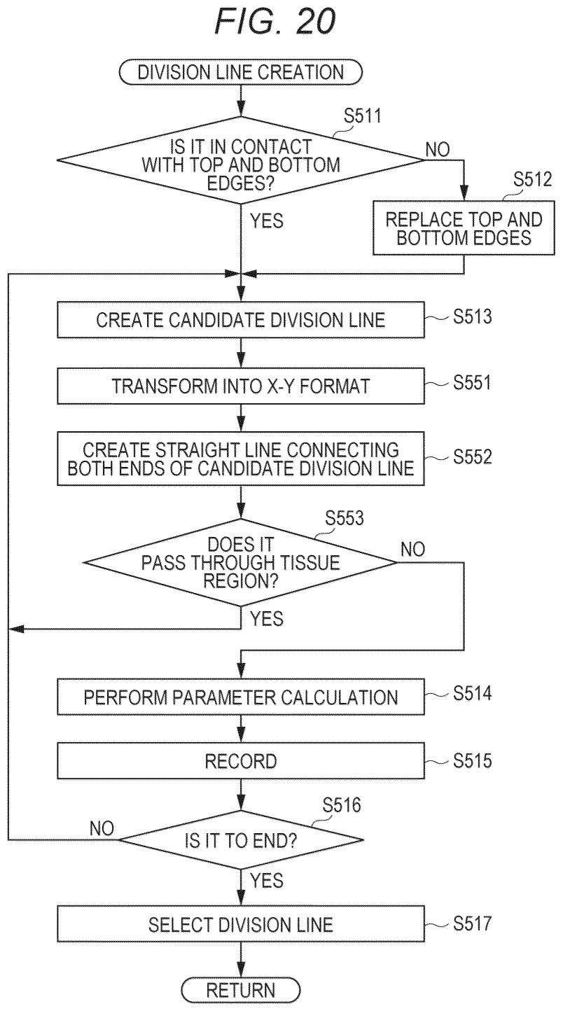

FIG. 20 is a flowchart for explaining the processing flow in a division line creation subroutine according to Modification 1-2. The division line creation subroutine is a subroutine for creating the division line 61 that divides the first lumen region 561 in an open state into the first region 571 on the side closer to the image-acquiring catheter 28 and the second region 572 on the side farther from the image-acquiring catheter 28. The subroutine in FIG. 20 is used instead of the subroutine described with reference to FIG. 14.

The processes from S511 to S513 are the same as the processes in the processing flow according to the program described with reference to FIG. 14, and therefore, explanation of them is not made herein. The control unit 201 converts the first classification data 51 on which the candidate division lines 62 are superimposed into an X-Y format (S551).

The control unit 201 creates a straight line connecting both ends of a candidate division line 62 converted into the X-Y format (S552). The control unit 201 determines whether the created straight line passes through the living tissue region 566 (S553). If it is determined that the created straight line passes through the living tissue region 566 (YES in S553), the control unit 201 returns to S513.

If it is determined that the created straight line does not pass through the living tissue region 566 (NO in S553), the control unit 201 calculates a predetermined parameter related to the candidate division line 62 (S514). The control unit 201 may calculate the parameter either in the R-T format or in the X-Y format. The control unit 201 may calculate the parameter in both the R-T format and the X-Y format. The processes that follow are the same as those in the processing flow according to the program described with reference to FIG. 14, and therefore, explanation of them is not made herein.

Images that are usually used by users in clinical practice are X-Y format images. According to the present modification, it is possible to automatically generate the division line 61 that matches the feeling of the user observing an X-Y image.

### Modification 1-3

The present modification relates to a method for selecting the division line 61 from a plurality of candidate division lines 62 in S517 in the flowchart described with reference to FIG. 20. Explanation of the same portions as those of Modification 1-2 is not made herein. In the present modification, in S514, the same parameter is calculated in both an R-T format and an X-Y format. After that, the division line 61 is selected on the basis of a result of calculation of the parameter calculated in the R-T format and the parameter calculated in the X-Y format.

A case where the lengths of candidate division lines 62 are used as parameters is now described as an example. The control unit 201 calculates an average value of the R-T length calculated on an R-T format image and the X-Y length calculated on an X-Y format image for each candidate division line 62. The average value is an arithmetic mean value or a geometric mean value, for example. For example, the control unit 201 selects the candidate division line 62 having the shortest average value, and determines the division line 61.

### Modification 1-4

In the present modification, feature points are extracted from the boundary line between the living tissue region 566 and the first lumen region 561, and candidate division lines 62 are created. Explanation of the same portions as those of the first embodiment is not made herein.

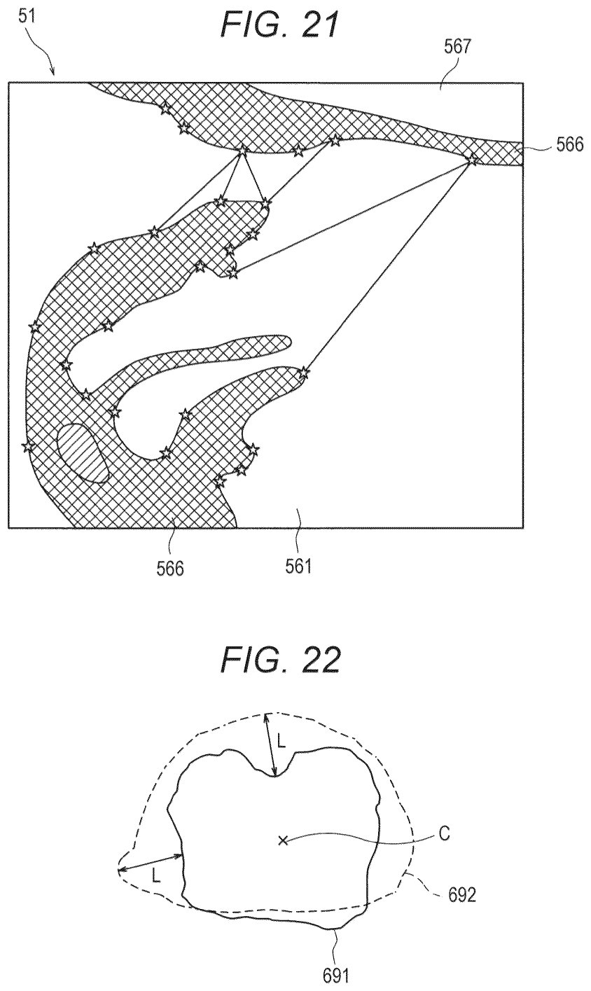

FIG. 21 is a diagram for explaining candidate division lines 62 according to Modification 1-4. Stars indicate feature points extracted from the boundary line between the living tissue region 566 and the first lumen region 561. The feature points are portions where the boundary line is curved, inflection points of the boundary line, and the like.

In the present modification, two feature points are connected to create a candidate division line 62. By limiting the start point and the end point of each candidate division line 62 to feature points, the process of creating the division line 61 can be speeded up.

### Modification 1-5

The present modification is a modification of the technique for quantifying the difference between the second classification data 52 and the third classification model 33 in S543 in the machine learning described with reference to FIG. 17. Explanation of the same portions as those of the first embodiment is not made herein.

FIG. 22 is a diagram for explaining machine learning according to Modification 1-5. A correct boundary line 691 indicated by a solid line represents the boundary line outside the first lumen region 561 in a case where the second classification data 52 is displayed in an X-Y format. Note that, for a region in which probabilities are allocated to the first lumen region 561 and the extra-luminal region 567 on the basis of the division line 61, a place where the probability of being the first lumen region 561 is 50% is defined as the boundary line of the first lumen region 561.

An output boundary line 692 indicated by a dashed line represents the boundary line outside the first lumen region 561 in the classification data being trained, which is obtained by inputting a two-dimensional image 58 to the third classification model 33 being trained and is output from the third classification model 33. C indicates the center of the two-dimensional image 58, which is the central axis of the image-acquiring catheter 28. L indicates the distance between the correct boundary line 691 and the output boundary line 692 in the scanning line direction of the image-acquiring catheter 28.

In S543, the control unit 201 adjusts the parameter of the third classification model 33 so that the average value of L measured at a total of 36 points in increments of 10 degrees becomes smaller, for example. The control unit 201 may adjust the parameter of the third classification model 33, for example, so that the maximum value of L becomes smaller.

### Second Embodiment

The present embodiment relates to a program that uses a two-dimensional image DB in which a large number of two-dimensional images 58 are recorded, instead of the first classification DB 41. The two-dimensional image DB is a database not having the first classification data field in the first classification DB 41 described with reference to FIG. 4. Explanation of the same portions as those of the first embodiment is not made herein.

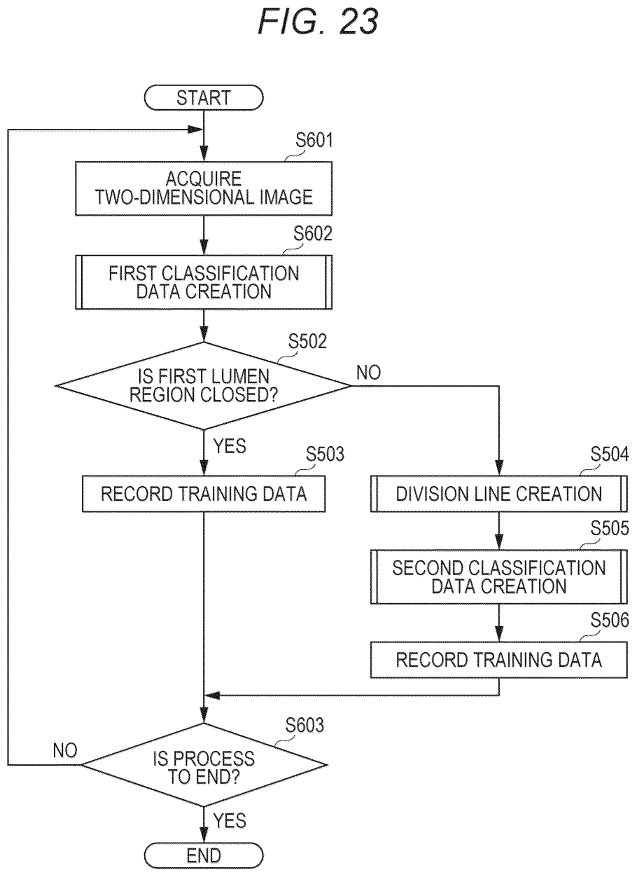

FIG. 23 is a flowchart for explaining the flow of processing according to a program of the second embodiment. The control unit 201 acquires one two-dimensional image from the two-dimensional image DB (S601). The control unit 201 starts a first classification data generation subroutine (S602). The first classification data generation subroutine is a subroutine for generating the first classification data 51 on the basis of the two-dimensional image 58. The flow of processing in the first classification data generation subroutine will be described later.

The control unit 201 determines whether the first lumen region 561 is in a closed state (S502). The processing flow up to S603 is the same as that according to the program of the first embodiment described with reference to FIG. 13, and therefore, explanation of the processing flow up to S603 is not made herein.

After S503 or S506 is completed, the control unit 201 determines whether to end the processing (S603). For example, in a case where the processing of all the records recorded in the two-dimensional image DB has been completed, the control unit 201 determines to end the processing. The control unit 201 may determine to end the processing in a case where the processing of a predetermined number of records has been completed.

If the control unit 201 determines not to end the processing (NO in S603), the control unit 201 returns to S601. If the control unit 201 determines to end the processing (YES in S603), the control unit 201 ends the processing.

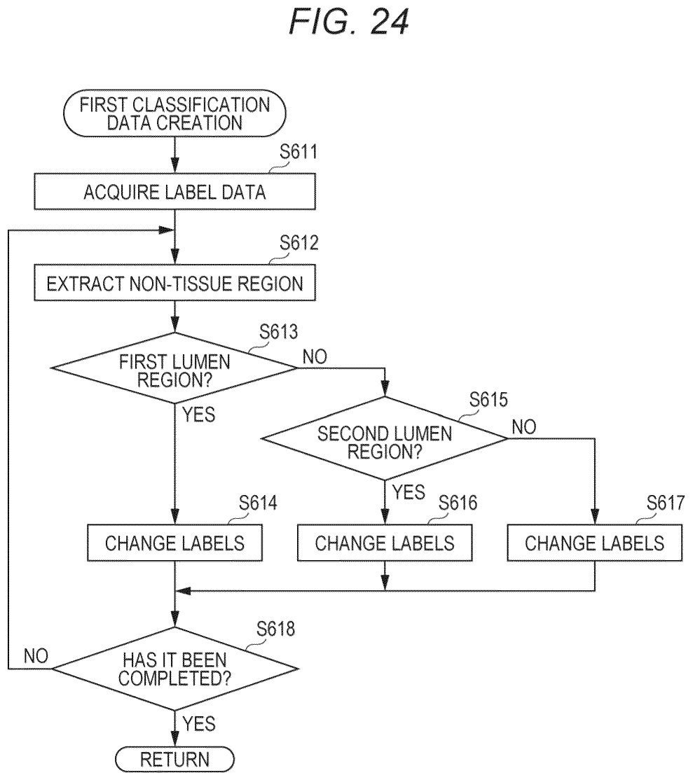

FIG. 24 is a flowchart for explaining the processing flow in the first classification data generation subroutine. The first classification data generation subroutine is a subroutine for generating the first classification data 51 on the basis of the two-dimensional image 58.

The control unit 201 inputs a two-dimensional image 58 to the label classification model 35, and acquires the label data 54 that is output (S611). The control unit 201 extracts, from the label data 54, a lump of a non-living tissue region 568 in which the label corresponding to the non-living tissue region 568 is recorded (S612).

The control unit 201 determines whether the extracted non-living tissue region 568 is a first lumen region 561 in contact with the edge on the side of the image-acquiring catheter 28 (S613). If the extracted non-living tissue region 568 is determined to be the first lumen region 561 (YES in S613), the control unit 201 changes the label corresponding to the non-living tissue region 568 extracted in S612, to the label corresponding to the first lumen region 561 (S614).

If the extracted non-living tissue region 568 is determined not to be the first lumen region 561 (NO in S613), the control unit 201 determines whether the extracted non-living tissue region 568 is a second lumen region 562 surrounded by the living tissue region 566 (S615). If the extracted non-living tissue region 568 is determined to be the second lumen region 562 (YES in S615), the control unit 201 changes the label corresponding to the non-living tissue region 568 extracted in S612, to the label corresponding to the second lumen region 562 (S616).

If the extracted non-living tissue region 568 is determined not to be the second lumen region 562 (NO in S615), the control unit 201 changes the label corresponding to the non-living tissue region 568 extracted in S612, to the label corresponding to an extra-luminal region 567 (S617).

After completion of S614, S616, or S617, the control unit 201 determines whether the processing of the non-living tissue region 568 included in the label data 54 acquired in S611 has been completed (S618). If it is determined that the processing has not been completed (NO in S618), the control unit 201 returns to S612. If it is determined that the processing has been completed (YES in S618), the control unit 201 ends the processing.

### Third Embodiment

The present embodiment relates to a catheter system 10 that generates a three-dimensional image in real time, using a three-dimensional scanning image-acquiring catheter 28. Explanation of the same portions as those of the first embodiment is not made herein.

FIG. 25 is a diagram for explaining the configuration of the catheter system 10 according to the third embodiment. The catheter system 10 can include an image processing apparatus 220, a catheter control device 27, a motor driving unit (MDU) 289, and the image-acquiring catheter 28. The image-acquiring catheter 28 is connected to the image processing apparatus 220 via the MDU 289 and the catheter control device 27.

The image processing apparatus 220 can include a control unit 221, a main storage device 222, an auxiliary storage device 223, a communication unit 224, a display unit 225, an input unit 226, and a bus. The control unit 221 is an arithmetic control device that executes a program according to the present embodiment. For the control unit 221, one or a plurality of CPUs or GPUs, a multi-core CPU, or the like is used. The control unit 221 is connected to each of the hardware components constituting the image processing apparatus 220 via the bus.

The main storage device 222 is a storage device such as an SRAM, a DRAM, or a flash memory. The main storage device 222 temporarily stores the information necessary in the middle of processing being performed by the control unit 221, and the program being executed by the control unit 221.

The auxiliary storage device 223 is a storage device such as an SRAM, a flash memory, a hard disk, or a magnetic tape. The auxiliary storage device 223 stores a label classification model 35, the program to be executed by the control unit 221, and various kinds of data necessary for executing the program. The communication unit 224 is an interface that conducts communication between the image processing apparatus 220 and a network. The label classification model 35 may be stored in an external mass storage device or the like connected to the image processing apparatus 220.

For example, the display unit 225 can be, for example, a liquid crystal display panel, an organic EL panel, or the like. The input unit 226 can be, for example, a keyboard, a mouse, or the like. The input unit 226 may be stacked on the display unit 225, to form a touch panel. The display unit 225 may be a display device connected to the image processing apparatus 220.

The image processing apparatus 220 is a general-purpose personal computer, a tablet, a large computing machine, or a virtual machine that runs on a large computing machine. The image processing apparatus 220 may be formed with a plurality of personal computers that perform distributed processing, or hardware such as a large computing machine. The image processing apparatus 220 may be formed with a cloud computing system. The image processing apparatus 220 and the catheter control device may constitute integrated hardware.

The image-acquiring catheter 28 includes a sheath 281, a shaft 283 inserted into the inside of the sheath 281, and a sensor 282 disposed at the distal end of the shaft 283. The MDU 289 rotates, advances, and retracts the shaft 283 and the sensor 282 inside the sheath 281.

The catheter control device 27 can generate one two-dimensional image 58 for each rotation of the sensor 282. Through an operation in which the MDU 289 rotates the sensor 282 while pulling or pushing the sensor 282, the catheter control device 27 continuously generates a plurality of two-dimensional images 58 substantially perpendicular to the sheath 281.

The control unit 221 successively acquires the two-dimensional images 58 from the catheter control device 27. The control unit 221 generates the first classification data 51 and the division line 61 on the basis of each two-dimensional image 58. The control unit 221 generates a three-dimensional image on the basis of a plurality of pieces of the first classification data 51 acquired in time series and the division line 61, and outputs the three-dimensional image to the display unit 225. In the above manner, so-called three-dimensional scanning is performed.

The operation of advancing and retracting the sensor 282 includes both of an operation of advancing and retracting the entire image-acquiring catheter 28, and an operation of advancing and retracting the sensor 282 inside the sheath 281. The advancing and retracting operation may be automatically performed at a predetermined speed by the MDU 289, or may be manually performed by the user.

Note that the image-acquiring catheter 28 is not necessarily of a mechanical scanning type that mechanically performs rotation, advancement, and retraction. For example, the image-acquiring catheter 28 may be an electronic radial scanning image-acquiring catheter 28 using the sensor 282 in which a plurality of ultrasound transducers is annularly disposed.

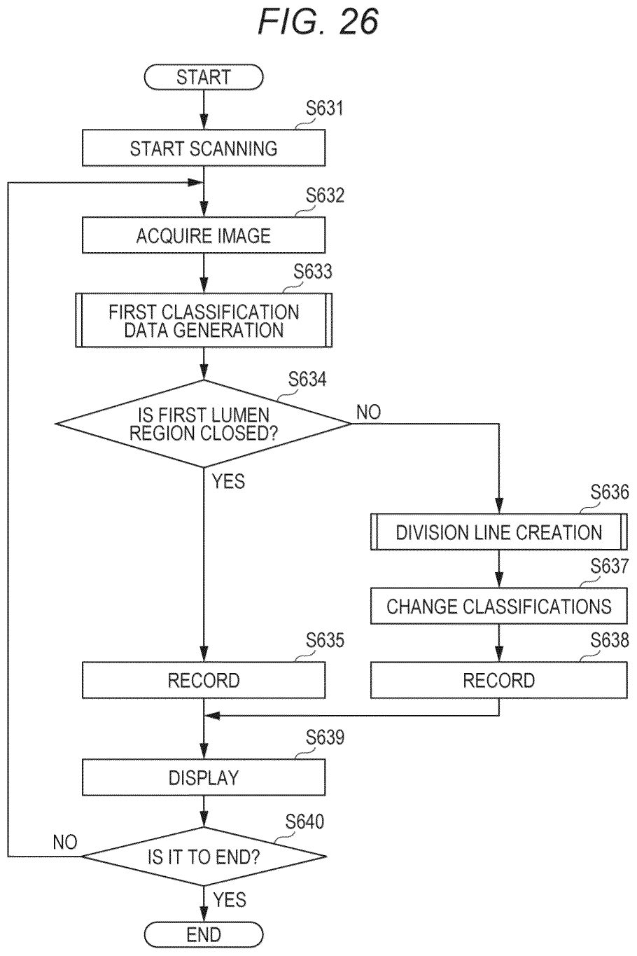

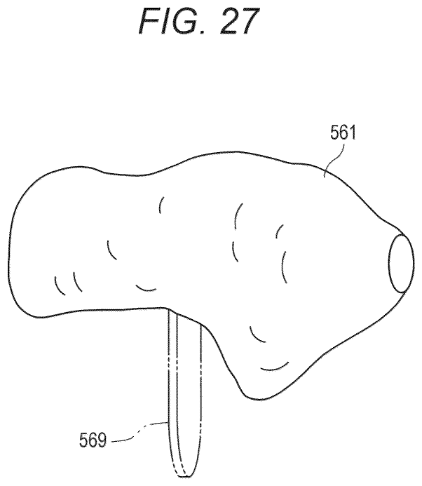

FIG. 26 is a flowchart for explaining the flow of processing according to a program of the third embodiment. In a case where an instruction to start three-dimensional scanning is received from the user, the control unit 221 executes the program that is now described with reference to FIG. 26.

The control unit 221 instructs the catheter control device 27 to start three-dimensional scanning (S631). The catheter control device 27 controls the MDU 289 to start three-dimensional scanning. The control unit 221 acquires one two-dimensional image 58 from the catheter control device 27 (S632). The control unit 221 starts the first classification data generation subroutine described with reference to FIG. 24 (S633). The first classification data generation subroutine is a subroutine for generating the first classification data 51 on the basis of the two-dimensional image 58.

The control unit 221 determines whether the first lumen region 561 is in a closed state (S634). If the first lumen region 561 is determined to be in a closed state (YES in S634), the control unit 221 records the first classification data 51 in the auxiliary storage device 223 or the main storage device 222 (S635).

If the first lumen region 561 is determined not to be in a closed state (NO in S634), the control unit 221 starts the division line creation subroutine described with reference to FIG. 14 or 20 (S636). The division line creation subroutine is a subroutine for creating the division line 61 that divides the first lumen region 561 in an open state into the first region 571 on the side closer to the image-acquiring catheter 28 and the second region 572 on the side farther from the image-acquiring catheter 28.

The control unit 221 changes the classification of the portion farther from the image-acquiring catheter 28 than the division line 61 in the first lumen region 561, to the extra-luminal region 567 (S637). The control unit 221 records the changed first classification data 51 in the auxiliary storage device 223 or the main storage device 222 (S638).

After the completion of S635 or S638, the control unit 221 displays, on the display unit 225, a three-dimensional image generated on the basis of the first classification data 51 recorded in time series (S639). The control unit 221 determines whether to end the processing (S640). For example, when a series of three-dimensional scanning operations has ended, the control unit 221 determines to end the processing.

If the control unit 221 determines not to end the processing (NO in S639), the control unit 221 returns to S632. If the control unit 221 determines to end the processing (YES in S639), the control unit 221 ends the processing.

The control unit 221 may record both the first classification data 51 generated in S633 and the first classification data 51 changed in S637 in the auxiliary storage device 223 or the main storage device 222. Instead of recording the changed first classification data 51, the control unit 221 may record the division line 61, and create changed first classification data 51 each time three-dimensional display is performed. The control unit 221 may receive, from the user, a selection as to which first classification data 51 is to be used in S639.

FIG. 27 is an example of display according to the third embodiment. A three-dimensional image of the first lumen region 561 extracted from the first classification data 51 is displayed. A correction region 569 indicated by an imaginary line is a region whose label has been changed from the first lumen region 561 to the extra-luminal region 567 in S636.

In a case where the first lumen region 561 is three-dimensionally displayed on the basis of the first classification data 51 generated in S633, the portion of the correction region 569 is also displayed. The correction region 569 is noise, and can inhibit the user from observing the portion hidden by the correction region 569.

Although a flowchart and an example screen are not shown, the control unit 221 receives operations such as changing the orientation, generating a cross section, changing the region to be displayed, and enlarging, reducing, or measuring the three-dimensional image illustrated in FIG. 27. The user can appropriately observe the three-dimensional image, and measure necessary data.

The user can rather easily observe the three-dimensional shape of the first lumen region 561 with the three-dimensional image in which the portion of the correction region 569 is erased using the program described with reference to FIG. 26. Further, the control unit 221 can accurately perform automatic measurement of the volume and the like of the first lumen region 561.

According to the present embodiment, it is possible to provide the catheter system 10 that displays a three-dimensional image with less noise in real time, using the three-dimensional image-acquiring catheter 28.

### Modification 3-1

The present modification relates to an image processing apparatus 220 that displays a three-dimensional image on the basis of a data set of two-dimensional images 58 recorded in time series. Explanation of the same portions as those of the third embodiment is not made herein. Note that, in the present modification, the catheter control device 27 is not necessarily connected to the image processing apparatus 220.

A data set of two-dimensional images 58 recorded in time series is recorded in the auxiliary storage device 223 or an external mass storage device. The data set may be, for example, a set of a plurality of two-dimensional images 58 generated on the basis of video data recorded during the past cases.

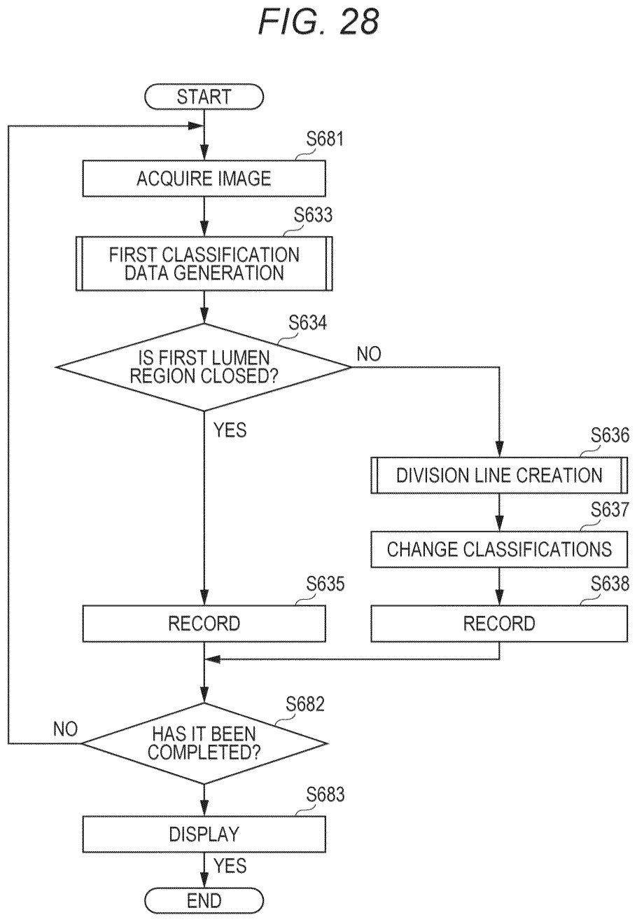

FIG. 28 is a flowchart for explaining the flow of processing according to a program of Modification 3-1. In a case where an instruction regarding the data set for performing three-dimensional display is received from the user, the control unit 221 executes the program that is now described with reference to FIG. 28.

The control unit 221 acquires one two-dimensional image 58 from the designated data set (S681). The control unit 221 starts the first classification data generation subroutine described with reference to FIG. 24 (S633). The processes from S634 to S638 that follow are similar to the processes according to the program of the third embodiment described with reference to FIG. 26, and therefore, explanation of them is not made herein.

After completion of S635 or S638, the control unit 221 determines whether the processing of the two-dimensional images 58 included in the designated data set has been completed (S682). If it is determined that the processing has not been completed (NO in S682), the control unit 221 returns to S681.

If it is determined that the processing has been completed (YES in S682), the control unit 221 displays, on the display unit 225, a three-dimensional image generated on the basis of the first classification data 51 and the changed first classification data 51 that are recorded in time series (S683).

According to the present modification, it is possible to provide the image processing apparatus 220 that displays a three-dimensional image with less noise, on the basis of the data set of two-dimensional images 58 recorded in time series.

Note that, instead of displaying a three-dimensional image in S683, the control unit 221 may record, in the auxiliary storage device 223, a data set in which the first classification data 51 and the changed first classification data 51 are recorded in time series, while also performing the processing in S683. The user can use the recorded data set, to observe the three-dimensional image as needed.

### Fourth Embodiment

The present embodiment relates to a catheter system 10 into which the third classification model 33 generated in the first embodiment or the second embodiment is installed. Explanation of the same portions as those of the third embodiment is not made herein.

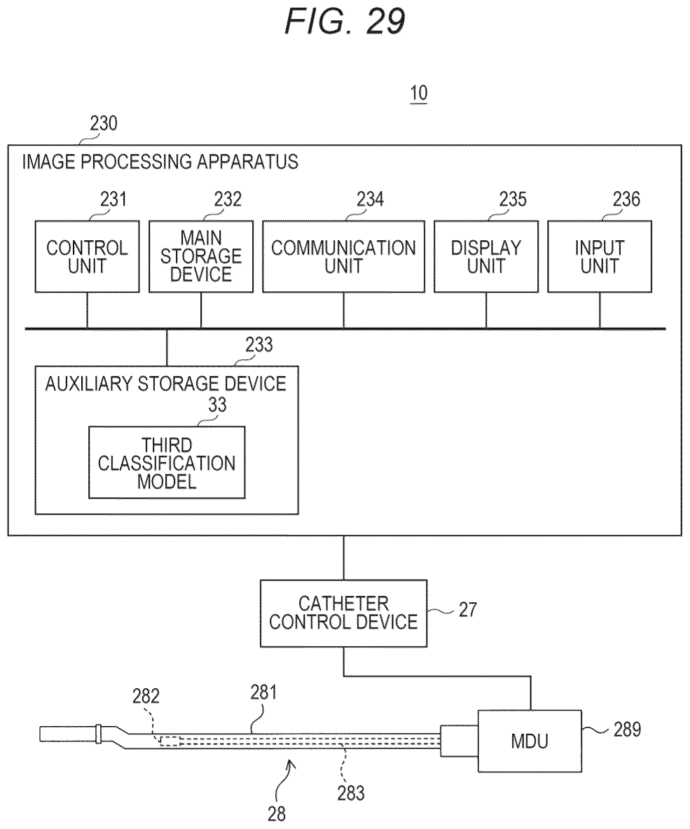

FIG. 29 is a diagram for explaining the configuration of the catheter system 10 according to the fourth embodiment. The catheter system 10 can include an image processing apparatus 230, a catheter control device 27, an MDU 289, and an image-acquiring catheter 28. The image-acquiring catheter 28 is connected to the image processing apparatus 230 via the MDU 289 and the catheter control device 27.

The image processing apparatus 230 can include a control unit 231, a main storage device 232, an auxiliary storage device 233, a communication unit 234, a display unit 235, an input unit 236, and a bus. The control unit 231 is an arithmetic control device that executes a program according to the present embodiment. For the control unit 231, one or a plurality of CPUs or GPUs, a multi-core CPU, or the like can be used. The control unit 231 is connected to each of the hardware components constituting the image processing apparatus 230 via the bus.

The main storage device 232 is a storage device such as an SRAM, a DRAM, or a flash memory. The main storage device 232 temporarily stores the information necessary in the middle of processing being performed by the control unit 231, and the program being executed by the control unit 231.

The auxiliary storage device 233 is a storage device such as an SRAM, a flash memory, a hard disk, or a magnetic tape. The auxiliary storage device 233 stores the third classification model 33, the program to be executed by the control unit 231, and various kinds of data necessary for executing the program. The communication unit 234 is an interface that conducts communication between the image processing apparatus 230 and a network. The third classification model 33 may be stored in an external mass storage device or the like connected to the image processing apparatus 230.

For example, the display unit 235 can be, for example, a liquid crystal display panel, an organic EL panel, or the like. For example, the input unit 236 is a keyboard, a mouse, or the like. The input unit 236 may be stacked on the display unit 235, to form a touch panel. The display unit 235 may be a display device connected to the image processing apparatus 230.

The image processing apparatus 230 is a general-purpose personal computer, a tablet, a large computing machine, or a virtual machine that runs on a large computing machine. The image processing apparatus 230 may be formed with a plurality of personal computers that perform distributed processing, or hardware such as a large computing machine. The image processing apparatus 230 may be formed with a cloud computing system. The image processing apparatus 230 and the catheter control device may constitute integrated hardware.

The control unit 231 sequentially acquires a plurality of two-dimensional images 58 obtained in time series from the catheter control device 27. The control unit 231 sequentially inputs the respective two-dimensional images 58 to the third classification model 33, to sequentially acquire the third classification data 53. The control unit 231 generates a three-dimensional image on the basis of a plurality of pieces of the third classification data 53 acquired in time series, and outputs the three-dimensional image to the display unit 235. In the above manner, so-called three-dimensional scanning is performed.

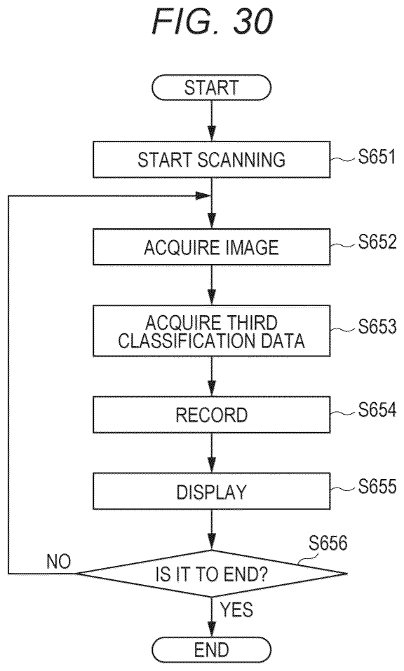

FIG. 30 is a flowchart for explaining the flow of processing according to a program of the fourth embodiment. In a case where an instruction to start three-dimensional scanning is received from the user, the control unit 231 executes the program that is now described with reference to FIG. 30.

The control unit 231 instructs the catheter control device 27 to start three-dimensional scanning (S651). The catheter control device 27 controls the MDU 289 to start three-dimensional scanning. The control unit 231 acquires one two-dimensional image 58 from the catheter control device 27 (S652).

The control unit 231 inputs the two-dimensional image 58 to the third classification model 33, and acquires the third classification data 53 that is output (S653). The control unit 231 records the third classification data 53 in the auxiliary storage device 233 or the main storage device 232 (S654).

The control unit 231 displays, on the display unit 235, a three-dimensional image generated on the basis of the third classification data 53 recorded in time series (S655). The control unit 231 determines whether to end the processing (S656). For example, when a series of three-dimensional scanning operations has ended, the control unit 231 determines to end the processing.

If the control unit 231 determines not to end the processing (NO in S656), the control unit 231 returns to S652. By repeating the processing in S653, the control unit 231 achieves the functions of a third classification data acquisition unit of the present embodiment that sequentially inputs a plurality of two-dimensional images obtained in time series to the third classification model 33, and sequentially acquires the third classification data 53 that is output. If the control unit 231 determines to end the processing (YES in S656), the control unit 231 ends the processing.

According to the present embodiment, it is possible to provide the catheter system 10 into which the third classification data 53 generated in the first embodiment or the second embodiment is installed. According to the present embodiment, it is possible to provide the catheter system 10 that realizes three-dimensional image display similar to that of the third embodiment, with a smaller calculation load than that of the third embodiment.

Note that both the third classification model 33 and the label classification model 35 may be recorded in the auxiliary storage device 233 or the auxiliary storage device 223 so that the user can select the processing according to the third embodiment and the processing according to the fourth embodiment.

### Modification 4-1

The present modification relates to an image processing apparatus 230 that displays a three-dimensional image on the basis of a data set of two-dimensional images 58 recorded in time series. Explanation of the same portions as those of the fourth embodiment is not made herein. Note that, in the present modification, the catheter control device 27 is not necessarily connected to the image processing apparatus 230.

A data set of two-dimensional images 58 recorded in time series is recorded in the auxiliary storage device 233 or an external mass storage device. The data set may be, for example, a set of a plurality of two-dimensional images 58 generated on the basis of video data recorded during the past cases.

The control unit 231 acquires one two-dimensional image 58 from the data set, inputs the two-dimensional image to the third classification model 33, and acquires the third classification data 53 that is output. The control unit 231 records the third classification data 53 in the auxiliary storage device 233 or the main storage device 232. After completing the processing of a series of data sets, the control unit 231 displays a three-dimensional image on the basis of the recorded third classification data 53.

According to the present modification, it is possible to provide the image processing apparatus 230 that displays a three-dimensional image with less noise, on the basis of the data set of two-dimensional images 58 recorded in time series.

Note that, instead of displaying a three-dimensional image, or together with displaying a three-dimensional image, the control unit 231 may record, in the auxiliary storage device 233, a data set in which the third classification data 53 is recorded in time series. The user can use the recorded data set, to observe the three-dimensional image as needed.

### Fifth Embodiment

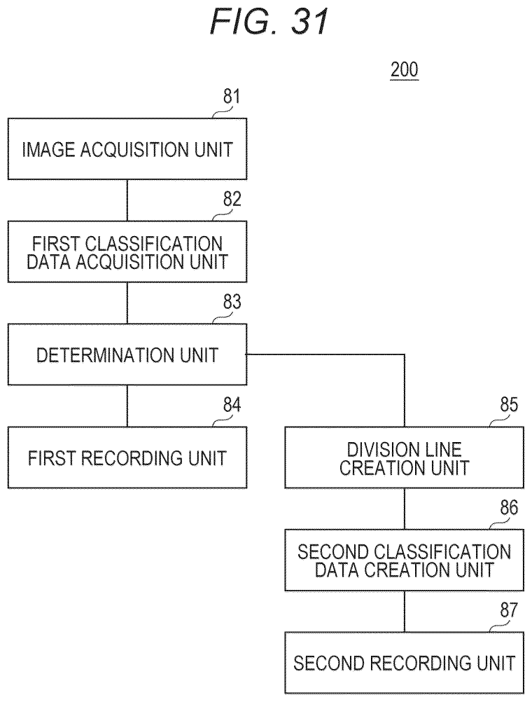

FIG. 31 is a functional block diagram of an information processing apparatus 200 according to a fifth embodiment. The information processing apparatus 200 includes an image acquisition unit 81, a first classification data acquisition unit 82, a determination unit 83, a first recording unit 84, a division line creation unit 85, a second classification data creation unit 86, and a second recording unit 87.

The image acquisition unit 81 acquires a two-dimensional image 58 acquired using an image-acquiring catheter 28. The first classification data acquisition unit 82 acquires first classification data 51 in which the two-dimensional image 58 is classified into a plurality of regions including a living tissue region 566, a first lumen region 561 into which the image-acquiring catheter 28 is inserted, and an extra-luminal region 567 outside the living tissue region 566.

In the two-dimensional image 58, the determination unit 83 determines whether the first lumen region 561 reaches an edge of the two-dimensional image 58. In a case where the determination unit 83 determines that the first lumen region 561 does not reach an edge of the two-dimensional image 58, the first recording unit 84 associates the two-dimensional image 58 with the first classification data 51, and records the two-dimensional image 58 and the first classification data 51 in a training DB 42.

In a case where the determination unit 83 determines that the first lumen region 561 reaches an edge, the division line creation unit 85 creates a division line 61 that divides the first lumen region 561 into a first region 571 into which the image-acquiring catheter 28 is inserted and a second region 572 that reaches the edge of the two-dimensional image 58. On the basis of the division line 61 and the first classification data 51, the second classification data creation unit 86 creates second classification data 52 in which a probability of being the first lumen region 561 and a probability of being the extra-luminal region 567 are allocated for each of the small regions constituting the first lumen region 561 in the first classification data 51. The second recording unit 87 associates the two-dimensional image 58 with the second classification data 52, and records the two-dimensional image 58 and the second classification data 52 in the training DB 42.

### Sixth Embodiment

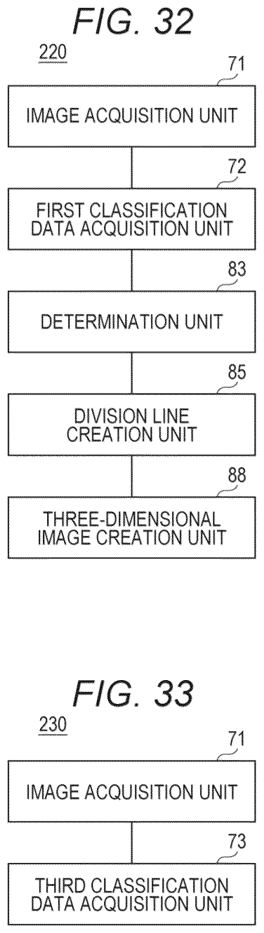

FIG. 32 is a functional block diagram of an image processing apparatus 220 according to a sixth embodiment. The image processing apparatus 220 includes an image acquisition unit 71, a first classification data acquisition unit 72, a determination unit 83, a division line creation unit 85, and a three-dimensional image creation unit 88.

The image acquisition unit 71 acquires a plurality of two-dimensional images 58 obtained in time series with an image-acquiring catheter 28. The first classification data acquisition unit 72 acquires a series of first classification data 51 in which the respective pixels constituting each two-dimensional image 58 of a plurality of two-dimensional images 58 are classified into a plurality of regions including a living tissue region 566, a first lumen region 561 into which the image-acquiring catheter 28 is inserted, and an extra-luminal region 567 outside the living tissue region 566.

In each two-dimensional image 58, the determination unit 83 determines whether the first lumen region 561 reaches an edge of the two-dimensional image 58. In a case where the determination unit 83 determines that the first lumen region 561 reaches an edge, the division line creation unit 85 creates a division line 61 that divides the first lumen region 561 into a first region 571 into which the image-acquiring catheter 28 is inserted and a second region 572 that reaches the edge of the two-dimensional image 58.

The three-dimensional image creation unit 88 creates a three-dimensional image by using a series of first classification data 51 in which the classification of the second region 572 has been changed to the extra-luminal region 567, or by using a series of first classification data 51 and processing the second region 572 as the same region as the extra-luminal region 567.

### Seventh Embodiment

FIG. 33 is a functional block diagram of an image processing apparatus 230 according to a seventh embodiment. The image processing apparatus 230 includes an image acquisition unit 71 and a third classification data acquisition unit 73.

The image acquisition unit 71 acquires a plurality of two-dimensional images 58 obtained in time series with an image-acquiring catheter 28. The third classification data acquisition unit 73 sequentially inputs the two-dimensional images 58 to a trained model 33 generated by the method described above, and sequentially acquires third classification data 53 that is output.

The technical features (components) described in the respective embodiments can be combined with each other, and new technical features can be formed by the combination.

The detailed description above describes to a learning model generation method, an image processing apparatus, an information processing apparatus, a training data generation method, and an image processing method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A learning model generation method comprising:
acquiring a two-dimensional image acquired with an image-acquiring catheter;
acquiring first classification data in which respective pixels constituting the two-dimensional image are classified into a plurality of regions including a living tissue region, a lumen region into which the image-acquiring catheter is inserted, and an extra-luminal region outside the living tissue region;
determining, in the two-dimensional image, whether the lumen region reaches an edge of the two-dimensional image;
when it is determined that the lumen region does not reach an edge of the two-dimensional image, associating the two-dimensional image with the first classification data, and recording the two-dimensional image associated with the first classification data in a training database;
when it is determined that the lumen region reaches an edge of the two-dimensional image,
creating a division line that divides the lumen region into a first region into which the image-acquiring catheter is inserted and a second region reaching an edge of the two-dimensional image;
creating second classification data in which a probability of being the lumen region and a probability of being the extra-luminal region are allocated for each of small regions constituting the lumen region in the first classification data, on a basis of the division line and the first classification data; and
associating the two-dimensional image with the second classification data, and recording the two-dimensional image associated with the second classification data in the training database; and
generating a learning model that outputs third classification data by machine learning using training data recorded in the training database when the two-dimensional image is input, respective pixels constituting the two-dimensional image being classified into a plurality of regions including the living tissue region, the lumen region, and the extra-lumen region in the third classification data.

2. The learning model generation method according to claim 1, further comprising:
determining whether the lumen region reaches an edge of the two-dimensional image on a basis of the first classification data.

3. The learning model generation method according to claim 1, further comprising:
determining whether the lumen region reaches an edge of the two-dimensional image on a basis of the two-dimensional image.

4. The learning model generation method according to claim 1, further comprising:
determining whether the lumen region reaches an edge of the two-dimensional image on a basis of an output of a reach determination model that outputs whether the lumen region reaches an edge of the two-dimensional image when the two-dimensional image is input to the reach determination model.

5. The learning model generation method according to claim 1, further comprising:
determining in the second classification data, a probability that each of the small regions is the lumen region and a probability that each of the small regions is the extra-luminal region on a basis of lengths of connecting lines connecting the small regions and the division line.

6. The learning model generation method according to claim 5, wherein the connecting lines pass only through a region classified as the lumen region in the first classification data.

7. The learning model generation method according to claim 5, wherein, in the second classification data, the probability that each of the small regions is the lumen region and the probability that each of the small regions is the extra-luminal region are defined by the following expressions:
where the small regions are closer to the image-acquiring catheter than the division line,
$$P 1 = \frac{1}{1 + {e}^{- \frac{L}{A}}}$$
$$P 2 = 1 - P 1$$
where the small regions are farther from the image-acquiring catheter than the division line,
$$P 1 = \frac{1}{1 + {e}^{\frac{L}{A}}}$$
$$P 2 = 1 - P 1$$
P1: probability that the small regions are in a lumen region;
P2: probability that the small regions are in an extra-luminal region;
L: length of the connecting line; and
A: constant.

8. The learning model generation method according to claim 1, further comprising:
determining each of the small regions in the second classification data to be the lumen region when the small region is closer to the image-acquiring catheter than the division line, and to be the extra-luminal region when the small region is farther from the image-acquiring catheter than the division line.

9. The learning model generation method according to claim 1, wherein
the division line is one or more of:
a line that passes only through the lumen region in an R-T format image in which the first classification data is shown in an R-T format, or in an X-Y format image in which the first classification data is shown in an X-Y format; and
a straight line in the R-T format image or the X-Y format image; and
the division line connects feature points extracted from a boundary line between a region classified as the living tissue region and a region classified as the lumen region in the R-T format image or the X-Y format image.

10. The learning model generation method according to claim 9, further comprising:
acquiring a plurality of candidate division lines satisfying a condition for the division line;
acquiring a length of each of the candidate division lines in the R-T format image; and
selecting the shortest candidate division line among the candidate division lines as the division line.

11. The learning model generation method according to claim 9, further comprising:
acquiring a plurality of candidate division lines satisfying a condition for the division line;
acquiring a length of each of the candidate division lines in the X-Y format image; and
selecting the shortest candidate division line among the candidate division lines as the division line.

12. The learning model generation method according to claim 9, further comprising:
acquiring a plurality of candidate division lines satisfying a condition for the division line;
acquiring an R-T length of each of the candidate division lines in the R-T format image, and an X-Y length of each of the candidate division lines in the X-Y format image; and
selecting the candidate division line having the shortest average value of the R-T length and the X-Y length as the division line.

13. The learning model generation method according to claim 12, wherein the average value is an arithmetic mean value or a geometric mean value.

14. The learning model generation method according to claim 1, wherein the machine learning includes:
acquiring a set of training data from the training database;
inputting a two-dimensional image included in the training data to a learning model being trained, to acquire third classification data that is output; and
repeating a process of adjusting a parameter of the learning model being trained, to reduce a difference between classification data recorded in the training data and the third classification data.

15. The learning model generation method according to claim 14, wherein
the difference is the number of pixels having different classifications in the classification data and in the third classification data, among respective pixels constituting the classification data recorded in the training data.

16. The learning model generation method according to claim 15, wherein
the difference is a distance between a correct boundary line related to a predetermined region in the classification data recorded in the training data and an output boundary line related to the predetermined region in the third classification data; and
the distance is a distance in a direction away from a center of the image-acquiring catheter.

17. An image processing apparatus comprising:
an image acquisition unit that acquires a plurality of two-dimensional images obtained in time series with an image-acquiring catheter; and
a third classification data acquisition unit configured to sequentially input the two-dimensional images to a trained model generated by the learning model generation method according to claim 1, and to sequentially acquire the third classification data that is output.

18. An image processing apparatus comprising:
an image acquisition unit configured to acquire a plurality of two-dimensional images obtained in time series with an image-acquiring catheter;
a first classification data acquisition unit configured to acquire a series of first classification data in which respective pixels constituting each two-dimensional image of the plurality of two-dimensional images are classified into a plurality of regions including a living tissue region, a lumen region into which the image-acquiring catheter is inserted, and an extra-luminal region outside the living tissue region;
a determination unit configured to determine whether the lumen region reaches an edge of each two-dimensional image, in each two-dimensional image of the plurality of two-dimensional images;
a division line creation unit configured to create a division line that divides the lumen region into a first region into which the image-acquiring catheter is inserted and a second region reaching an edge of the two-dimensional image, when the determination unit determines that the lumen region reaches an edge of the two-dimensional image; and
a three-dimensional image creation unit configured to create a three-dimensional image by using the series of first classification data in which a classification of the second region has been changed to the extra-luminal region, or by using the series of first classification data and processing the second region as the same region as the extra-luminal region.

19. The information processing apparatus according to claim 18, further comprising:
a first recording unit configured to associate the two-dimensional image with the first classification data and to record the two-dimensional image associated with the first classification data in a training database, when the determination unit determines that the lumen region does not reach an edge of the two-dimensional image;
a second classification data creation unit configured to create second classification data in which a probability of being the lumen region and a probability of being the extra-luminal region are allocated for each of small regions constituting the lumen region of the first classification data, on a basis of the division line and the first classification data, when the determination unit determines that the lumen region reaches an edge of the two-dimensional image; and
a second recording unit configured to associate the two-dimensional image with the second classification data, and records the two-dimensional image associated with the second classification data in the training database, when the determination unit determines that the lumen region reaches an edge of the two-dimensional image.

20. An image processing method comprising:
acquiring a plurality of two-dimensional images obtained in time series with an image-acquiring catheter;
acquiring a series of first classification data in which respective pixels constituting each two-dimensional image of the plurality of two-dimensional images are classified into a plurality of regions including a living tissue region, a lumen region into which the image-acquiring catheter is inserted, and an extra-luminal region outside the living tissue region;
determining whether the lumen region reaches an edge of each two-dimensional image, in each two-dimensional image of the plurality of two-dimensional images;
creating a division line that divides the lumen region into a first region into which the image-acquiring catheter is inserted and a second region reaching an edge of the two-dimensional image, when it is determined that the lumen region reaches an edge of the two-dimensional image; and
creating a three-dimensional image by using the series of first classification data in which a classification of the second region has been changed to the extra-luminal region, or by using the series of first classification data and processing the second region as the same region as the extra-luminal region.
